(19) United States
(12) Patent Application Publication
(10) Pub. No.: US 2022/0089710 A1
(43) Pub. Date: Mar. 24, 2022

(54) Method of Treating Signs and Symptoms of Osteoarthritis

(71) Applicant: Pfizer Inc., New York, NY (US)

(72) Inventors: Christine Ruth WEST, Williamston, MI (US); Mark Thomas BROWN, Kalamazoo, MI (US)

(21) Appl. No.: 17/423,690

(22) PCT Filed: Jan. 27, 2020
(86) PCT No.: PCT/IB2020/050611

§ 371 Date: Jul. 16, 2021

Related U.S. Application Data
(60) Provisional application No. 62/797,537, filed on Jan. 28, 2019.
(60) Provisional application No. 62/835,297, filed on Apr. 17, 2019.
(60) Provisional application No. 62/851,988, filed on May 23, 2019.
(60) Provisional application No. 62/923,663, filed on Oct. 21, 2019.
(60) Provisional application No. 62/947,113, filed on Dec. 12, 2019.
(60) Provisional application No. 62/949,777, filed on Dec. 18, 2019.

(51) Int. Cl. C07K 16/22; A61P 19/02; A61B 6/00; A61K 9/00

(52) U.S. Cl. CPC C07K 16/22; A61K 9/0019; A61B 6/505; A61P 19/02

(57) ABSTRACT

The present invention relates to the treatment of signs and symptoms of osteoarthritis with an anti-nerve growth factor (NGF) antibody.

### FIELD

The present invention relates to the treatment of signs and symptoms of osteoarthritis with an anti-nerve growth factor (NGF) antibody.

### BACKGROUND

Osteoarthritis (OA) is a major cause of pain and locomotor disability (McAlindon et al. Osteoarthritis Cartilage. 2014; 22(3):363-388). Despite a number of treatment options and guidelines for management of pain associated with OA, many patients report dissatisfaction with or the need to change medications because adequate pain control is not achieved (McAlindon et al 2014, Hochberg et al. Arthritis Care Res (Hoboken). 2012; 64(4):465-474; Zhang et al. Osteoarthritis Cartilage. 2008; 16(2):137-162. Non-steroidal anti-inflammatory drugs (NSAIDs) and opioids are standard pharmacologic treatments for OA pain, but these are often associated with increased risk of adverse events (AEs), including gastrointestinal and cardiovascular AEs, multi-organ failure, and potential for dependence or addiction (McAlindon et al; Hochberg et al; Zhang et al). The elderly and/or patients with diabetes, in particular, are more susceptible to these AEs than the rest of the population (Kim et al. BMJ open diabetes research & care. 2015; 3(1):e000133; Sowers et al. Arch Intern Med. 2005; 165(2):161-168; Wehling et al. European journal of clinical pharmacology. 2014; 70(10):1159-1172).

Development of novel pharmacologic therapies targeting the function of key pain modulators may provide new treatment options with improved efficacy and/or safety. Nerve growth factor (NGF) is a neurotrophin and key mediator of pain, with a demonstrated role in pain signal transduction and pathophysiology. Tanezumab is a humanized anti-NGF monoclonal antibody that has high specificity and affinity for NGF, thereby blocking binding of NGF to its receptors, TrkA and p75 (Abdiche et al. Protein Sci. 2008; 17(8):1326-1335; Hefti et al. Trends Pharmacol Sci. 2006; 27(2):85-91; Mantyh et al. Anesthesiology. 2011; 115(1):189-204). In randomized clinical trials in patients with chronic pain conditions (OA and chronic low back pain), tanezumab provided clinically meaningful improvements by significantly reducing pain and improving physical function and Patient's Global Assessment (PGA) of OA (Balnescu et al. Ann Rheum Dis. 2014; 73(9):1665-1672; Brown et al. J Neurol Sci. 2014; 345(1-2):139-147; Brown et al. J Pain. 2012; 13(8):790-798; Brown et al. Arthritis Rheum. 2013; 65(7):1795-1803; Ekman et al. J Rheumatol. 2014; 41(11):2249-2259; Evans et al. J Urol. 2011; 185(5):1716-1721; Gimbel et al. Pain. 2014; 155(9):1793-1801; Katz et al. Pain. 2011; 152(10):2248-2258; Kivitz et al. Pain. 2013; 154(7):1009-1021; Lane et al. N Engl J Med. 2010; 363(16):1521-1531; Nagashima et al. Osteoarthritis Cartilage. 2011; 19(12):1405-1412; Schnitzer et al. Ann Rheum Dis. 2015; 74(6):1202-1211; Schnitzer et al. Osteoarthritis Cartilage. 2011; 19(6):639-646; Spierings et al. Pain. 2013; 154(9):1603-1612; Spierings et al. Pain. 2014; 155(11):2432-2433). During conduct of late-phase development studies, unexpected AEs requiring total joint replacement led the US Food and Drug Administration to impose a partial clinical hold on all NGF-inhibitor therapies in development (for all indications except for cancer pain). A blinded Adjudication Committee reviewed and adjudicated the joint-related AEs and determined tanezumab treatment in higher doses and in combination with NSAIDs was associated with an increase in rapidly progressive OA (Hochberg et al. Arthritis Rheumatol. 2016; 68(2):382-391). The partial clinical hold was subsequently lifted and risk-mitigation strategies have been incorporated into anti-NGF antibody trial design.

Safety is a concern regarding long-term therapy with opioids or NSAIDs. Opioids are associated with a variety of common adverse effects including somnolence, sedation, nausea, vomiting, dizziness, dry mouth, pruritus, smooth muscle spasm, urinary retention, and constipation. This is due to the presence of opioid receptors, and a role for opioid receptor signaling, in a variety of structures both within and outside of the CNS, such as the GI tract (e.g., constipation), vestibular system (e.g., nausea and dizziness), and the medulla (e.g., vomiting). Respiratory depression is a less common AE with chronic opioid use, but this potentially serious event is mediated through activation of μ-opioid receptors located in respiratory centers of the brainstem and/or structures that signal CO₂ retention to the brainstem. Finally, traditional μ-opioid receptor agonists activate dopamine signaling in the mesolimbic system by inhibiting release of GABA from inhibitory interneurons, which can produce euphoria and lead to a powerful rewarding state in some patients. Unmonitored opioid use can result in the development of addiction and it is estimated that 11.5 million people abuse opioids in the US, and deaths due to opioid overdose have risen over the past decade, with approximately 42,000 deaths per year.

### SUMMARY

The invention disclosed herein is directed to treatment of signs and symptoms of osteoarthritis in patients who have a history of inadequate pain relief or intolerance to analgesic therapy including opioids.

Accordingly, in one aspect, the invention provides a method for treating signs and symptoms of osteoarthritis (OA) in a patient, the method comprising administering to the patient an anti-nerve growth factor (NGF) antibody at a dose of 2.5 mg every 8 weeks via subcutaneous injection; wherein the patient has a history of inadequate pain relief or intolerance to analgesic therapy and the treatment with the anti-NGF antibody effectively improves signs and symptoms of OA by at least 16 weeks after start of treatment with the anti-NGF antibody.

In a further aspect, the invention provides a method for treating signs and symptoms of osteoarthritis (OA) in a patient, the method comprising administering to the patient an anti-nerve growth factor (NGF) antibody at a dose of 5 mg every 8 weeks via subcutaneous injection; wherein the patient has a history of inadequate pain relief or intolerance to analgesic therapy and the treatment with the anti-NGF antibody effectively improves signs and symptoms of OA by at least 16 weeks after start of treatment with the anti-NGF antibody.

In some embodiments, the anti-NGF antibody is tanezumab.

In some embodiments the treatment effectively reduces pain associated with OA. In some embodiments the pain is moderate to severe chronic pain associated with OA.

In some embodiments, the treatment improves OA signs and symptoms as measured by WOMAC Pain subscale, WOMAC Physical Function subscale and/or Patient Global Assessment of OA (PGA-OA).

In some embodiments, the treatment effectively improves signs and symptoms of OA by at least 24 weeks or by at least 56 weeks after start of treatment.

In some embodiments the treatment improves WOMAC Pain, WOMAC Physical Function and/or PGA-OA compared to a baseline value prior to or at start of treatment.

In some embodiments the treatment improves OA signs and symptoms compared to analgesic therapy. In some embodiments, the analgesic therapy may include an NSAID and/or an opioid. In some embodiments the analgesic therapy does not include administration of an opioid. In some embodiments the analgesic therapy does not include the administration of an NSAID. In some embodiments, the treatment improves OA signs and symptoms compared to the OA signs and symptoms before start of treatment with the NGF antibody.

In some embodiments, the treatment further improves one or more clinical measures selected from a) reduction in WOMAC Pain subscale of 50% at week 16 and/or at week 24 of treatment; b) reduction in WOMAC Pain subscale from baseline to week 2 of treatment; or c) reduction in average pain score in index joint from baseline at week 1 of treatment.

In some embodiments the patient has a history of inadequate pain relief or intolerance to analgesic therapy, which can include NSAIDs, tramadol or opioids. In some embodiments, the patient has a history of inadequate pain relief or intolerance to at least two, at least three, or at least four different classes of analgesics. In some embodiments, the patient has a history of inadequate pain relief or intolerance to at least two, at least three, at least four analgesics. In some embodiments the patient has a history of unwillingness to take one or more analgesics, in an embodiment an opioid analgesic, in prior treatment. In some embodiments the patient was unable to take an analgesic due to contraindication. In some embodiments the patient was unable to take tramadol or opioids due to contraindication. In some embodiments the patient is diagnosed with opioid addiction. The rationale for choice of this population is to optimize the potential benefit-risk relationship for patients to be treated by selecting patients who have pain that is more severe or treatment-resistant and who have limited treatment options remaining.

In some embodiments, the patient was previously treated with the analgesic therapy prior to administering the anti-NGF antibody.

In some embodiments, the patient has a history of treatment with at least one, at least two, at least three, at least four or at least five analgesic therapies. The analgesic may be from the same or different class of analgesic.

In some embodiments, the analgesic therapy comprises the administration of an opioid to the patient. In some embodiments the analgesic therapy comprises the administration of tramadol to the patient. In some embodiments the analgesic therapy comprises the administration of an NSAID to the patient. In some embodiments, the NSAID is selected from naproxen, celecoxib or diclofenac.

In some embodiments, the treatment averts opioid addiction in the patient. In some embodiments, the treatment with the anti-NGF antibody avoids administration of an opioid and averts opioid addiction.

In some embodiments the patient has a history of addiction to analgesics. In some embodiments, the patient has a history of addiction to opioids. In some embodiments, the patient has a history of addiction to tramadol.

In some embodiments, the analgesic may be selected from opioids, NSAIDs, acetaminophen. In some embodiments the NSAID is selected from ibuprofen, naproxen, naprosyn, diclofenac, ketoprofen, tolmetin, slindac, mefenamic acid, meclofenamic acid, diflunisal, flufenisal, piroxim, sudoxicam, isoxicam; a COX-2 inhibitor selected from celecoxib, rofecoxib, DUP-697, flosulide, meloxicam, 6-methoxy-2 naphthylacetic acid, MK-966, nabumetone, nimesulide, NS-398, SC-5766, SC-58215, T-614; or combinations thereof. In some embodiments, the opioid may be any compound exhibiting morphine-like biological activity. In some embodiments, the opioid analgesic is selected from: tramadol, morphine, codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine; or combinations thereof.

In some embodiments, the patient is not administered an NSAID during the treatment with the anti-NGF antibody. In some embodiments, the patient is not administered an NSAID for 16 weeks after the last dose of the antibody.

In some embodiments the patient has moderate to severe osteoarthritis pain.

In some embodiments, the patient has been diagnosed with osteoarthritis for at least two, at least three, at least four, at least five, at least six years prior to treatment with the anti-NGF antibody.

In some embodiments, the OA is of the hip, knee, shoulder or hand. In some embodiments, OA is of the hip or knee.

In some embodiments, the anti-NGF antibody is administered for at least two, three, four, five, six or more doses at eight weekly intervals.

In some embodiments the 2.5 mg dose is increased to 5 mg after at least one eight week dose.

In some embodiments, patients have non-response if WOMAC Pain is reduced by <30%; moderate response if WOMAC Pain is reduced by ≥30% but <50%; and substantial response if WOMAC Pain is reduced by ≥50% from baseline. In some embodiments, the level of reduction is assessed between two time points during treatment.

In some embodiments, patients having non-response with 2.5 mg dose receive increased subsequent doses. In some embodiments, the dose is increased to 5 mg.

In some embodiments, patients not having satisfactory clinical response after receiving two doses do not receive further doses.

In some embodiments, the patient, prior to administering the anti-NGF antibody, has a) WOMAC Pain subscale measure of in the osteoarthritic joint; b) WOMAC Physical Function subscale measure of in the osteoarthritic joint; and/or c) a PGA-OA measure of fair, poor, or very poor.

In some embodiments, the patient, prior to administering the anti-NGF antibody, has a Kellgren-Lawrence x-ray grade of ≥2. In some embodiments, the patient has a Kellgren-Lawrence grade of 2, 3 or 4. In some embodiments the patient has severe radiographic osteoarthritis with a Kellgren-Lawrence grade of 4 in the index joint.

In some embodiments, the patient, prior to administering the anti-NGF antibody, has been receiving a stable dose regimen of an NSAID. In some embodiments, the patient, prior to administering the anti-NGF antibody, has not been receiving an NSAID. In some embodiments, the patient has had a prior adverse event following administration of an NSAID.

In some embodiments, the patient is subjected to radiographic assessment of the osteoarthritic joint prior to starting treatment with the anti-NGF antibody. In some embodiments, if radiographic assessment identified rapidly progressive osteoarthritis of the joint, the patient is excluded from the treatment with the anti-NGF antibody.

In some embodiments, the method further comprises conducting a radiographic assessment of the osteoarthritic joint at regular intervals during treatment with the anti-NGF antibody.

In some embodiments, a patient may be excluded from treatment, before or during treatment, with the anti-NGF antibody if there is radiographic evidence of any of the following conditions in any screening radiograph as determined by a central radiology reviewer and as defined in an imaging atlas: excessive malalignment of the knee, severe chondrocalcinosis; other arthropathies (e.g., rheumatoid arthritis), systemic metabolic bone disease (e.g., pseudogout, Paget's disease; metastatic calcifications), large cystic lesions, primary or metastatic tumor lesions, stress or traumatic fracture.

In some embodiments, a patient may be excluded from treatment, before or during treatment, with the anti-NGF antibody if there is radiographic evidence of any of the following conditions as determined by the central radiology reviewer and as defined in an imaging atlas at screening: 1) rapidly progressive osteoarthritis, 2) atrophic or hypotrophic osteoarthritis, 3) subchondral insufficiency fractures, 4) spontaneous osteonecrosis of the knee (SPONK), 5) osteonecrosis, or 6) pathologic fracture.

In some embodiments the patent is monitored for the development of signs and symptoms of rapidly progressive osteoarthritis prior to each dose. In some embodiments monitoring include radiographic assessment (such as X-ray).

In some embodiments the radiographic assement is performed annually during treatment. The radiographic assessment may be a bilateral assessment of the hip and/or knee. In some embodiments, symptoms of rapidly progressive osteoarthritis may include new onset, severe persistent pain or swelling in a joint. In some embodiments, treatment is discontinued if a patient develops rapidly progressive osteoarthritis.

In some embodiments, the anti-NGF antibody comprises three CDRs from the variable heavy chain region having the sequence shown in SEQ ID NO: 1 and three CDRs from the variable light chain region having the sequence shown in SEQ ID NO: 2. In some embodiments, the anti-NGF antibody comprises a HCDR1 having the sequence shown in SEQ ID NO:3, a HCDR2 having the sequence shown in SEQ ID NO:4, a HCDR3 having the sequence shown in SEQ ID NO:5, a LCDR1 having the sequence shown in SEQ ID NO:6, a LCDR2 having the sequence shown in SEQ ID NO:7, and a LCDR3 having the sequence shown in SEQ ID NO:8. In some embodiments, the anti-NGF antibody comprises a variable heavy chain region having the sequence shown in SEQ ID NO: 1 and a variable light chain region having the sequence shown in SEQ ID NO: 2. In some embodiments, the anti-NGF antibody comprises a heavy chain having the sequence shown in SEQ ID NO: 9 and a light chain having the sequence shown in SEQ ID NO: 10. In some embodiments, the C-terminal lysine (K) of the heavy chain amino acid sequence of SEQ ID NO: 9 is optional. Thus, in some embodiments the heavy chain amino acid sequence lacks the C-terminal lysine (K) and has the sequence shown in SEQ ID NO: 11.

In some embodiments, the method can further comprise administering an effective amount of a second therapeutic agent.

Also provided is an anti-NGF antibody for use in a method for treating signs and symptoms of osteoarthritis (OA) in a patient, the method comprising administering to the patient an anti-nerve growth factor (NGF) antibody at a dose of 2.5 mg or 5 mg every 8 weeks via subcutaneous injection; wherein the patient has a history of inadequate pain relief or intolerance to analgesic therapy, and the treatment with the anti-NGF antibody effectively improves signs and symptoms of OA by at least 16 weeks after start of treatment with the anti-NGF antibody.

Also provided is the use of an anti-NGF antibody in the manufacture of a medicament for the treatment of signs and symptoms of osteoarthritis (OA) in a patient, the treatment comprising administering to the patient an anti-nerve growth factor (NGF) antibody at a dose of 2.5 mg every 8 weeks via subcutaneous injection; wherein the patient has a history of inadequate pain relief or intolerance to analgesic therapy, and the treatment with the anti-NGF antibody effectively improves signs and symptoms of OA by at least 16 weeks after start of treatment with the anti-NGF antibody.

Also provided is an anti-NGF antibody for use in a method for treating signs and symptoms of osteoarthritis (OA) in a patient, the method comprising administering to the patient an anti-nerve growth factor (NGF) antibody at a dose of 2.5 mg or 5 mg every 8 weeks via subcutaneous injection; wherein the patient has a history of inadequate pain relief or intolerance to analgesic therapy, and the use of the anti-NGF antibody in the treatment is to effectively improve signs and symptoms of OA by at least 16 weeks after start of treatment with the anti-NGF antibody.

Also provided is the use of an anti-NGF antibody in the manufacture of a medicament for the treatment of signs and symptoms of osteoarthritis (OA) in a patient, the treatment comprising administering to the patient an anti-nerve growth factor (NGF) antibody at a dose of 2.5 mg every 8 weeks via subcutaneous injection; wherein the patient has a history of inadequate pain relief or intolerance to analgesic therapy, and the use of the anti-NGF antibody in the treatment is to effectively improve signs and symptoms of OA by at least 16 weeks after start of treatment with the anti-NGF antibody.

### BRIEF DESCRIPTION OF THE FIGURES/DRAWINGS

FIG. 1 is a study outline for the study described in Example 1.

FIG. 2 shows change from baseline to Week 16 in the WOMAC Pain, WOMAC Physical Function, and PGA-OA for the study described in Example 1.

FIG. 3 shows changes in WOMAC Pain, WOMAC Physical Function and Average Daily Pain in the Index Joint Scores during the treatment period for the study described in Example 1.

FIG. 4 shows WOMAC Pain Responder rates at week 16 for the study described in Example 1.

FIG. 5 shows WOMAC Pain responder rates at week 16 in non-responders at week 8 for the study described in Example 1.

FIG. 6 shows change, from baseline, in WOMAC Pain, WOMAC Physical Function, and PGA-OA scores at week 24 for the study described in Example 2.

FIG. 7 shows the change from baseline for the WOMAC Pain Subscale up to Week 24.

FIG. 8 shows the change from baseline for the WOMAC Physical Function Subscale up to Week 24.

FIG. 9 shows the change from baseline for the Patient Global Assessment of OA up to Week 24.

FIG. 10 shows the change from baseline for the WOMAC Pain Subscale Up to Week 56 for the study described in Example 3.

FIG. 11 shows the change from baseline for the WOMAC Physical Function Subscale up to Week 56 for the study described in Example 3.

FIG. 12 shows the change from baseline for the Patient Global Assessment of OA up to week 56 for the study described in Example 3.

### DETAILED DESCRIPTION

The invention disclosed herein is directed to treatment of signs and symptoms of osteoarthritis in patients who have a history of inadequate pain relief or intolerance to analgesic therapy.

Accordingly, in one aspect, the invention provides a method for treating signs and symptoms of osteoarthritis (OA) in a patient, the method comprising administering to the patient an anti-nerve growth factor (NGF) antibody at a dose of 2.5 mg every 8 weeks via subcutaneous injection; wherein the patient has a history of inadequate pain relief or intolerance to analgesic therapy and the treatment with the anti-NGF antibody effectively improves signs and symptoms of OA by at least 16 weeks after start of treatment with the anti-NGF antibody.

In a further aspect, the invention provides a method for treating signs and symptoms of osteoarthritis (OA) in a patient, the method comprising administering to the patient an anti-nerve growth factor (NGF) antibody at a dose of 5 mg every 8 weeks via subcutaneous injection; wherein the patient has a history of inadequate pain relief or intolerance to analgesic therapy and the treatment with the anti-NGF antibody effectively improves signs and symptoms of OA by at least 16 weeks after start of treatment with the anti-NGF antibody.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definitions

The following terms, unless otherwise indicated, shall be understood to have the following meanings:

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)₂, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy-chain constant regions that correspond to the different classes of immunoglobulins are called alpha, delta, epsilon, gamma, and mu, respectively. The subunit structures and three-dimensional configurations of different classes of immunoglobulins are well known.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “ABM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As known in the art, a “constant region” of an antibody refers to the constant region of the antibody light chain or the constant region of the antibody heavy chain, either alone or in combination.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

As used herein, “humanized” antibody refers to forms of non-human (e.g. murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

In some instances, Fv framework region (FR) residues of the human immunoglobulin are replaced by corresponding non-human residues. Furthermore, the humanized antibody may include residues that are found neither in the recipient antibody nor in the imported CDR or framework sequences, but are included to further refine and optimize antibody performance. In general, the humanized antibody will include substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will include at least a portion of an immunoglobulin constant region or domain (Fc), typically that of a human immunoglobulin. In some aspects of the invention the antibodies have Fc regions modified as described in PCT International Publication No. WO 99/58572. Other forms of humanized antibodies have one or more CDRs (CDR L1, CDR L2, CDR L3, CDR H1, CDR H2, or CDR H3) which may be altered with respect to the original antibody, which are also termed one or more CDRs “derived from” one or more CDRs from the original antibody.

Humanization can be essentially performed following the method of Winter and co-workers (Jones et al. Nature 321:522-525 (1986); Riechmann et al. Nature 332:323-327 (1988); Verhoeyen et al. Science 239:1534-1536 (1988)), by substituting rodent or mutant rodent CDRs or CDR sequences for the corresponding sequences of a human antibody. See also U.S. Pat. Nos. 5,225,539; 5,585,089; 5,693,761; 5,693,762; 5,859,205; which are incorporated herein by reference in its entirety. In some instances, residues within the framework regions of one or more variable regions of the human immunoglobulin are replaced by corresponding non-human residues (see, for example, U.S. Pat. Nos. 5,585,089; 5,693,761; 5,693,762; and 6,180,370). Furthermore, humanized antibodies may include residues that are not found in the recipient antibody or in the donor antibody. These modifications are made to further refine antibody performance (e.g., to obtain desired affinity). In general, the humanized antibody will include substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will include at least a portion of an immunoglobulin constant region (Fc), typically that of a human immunoglobulin. For further details see Jones et al. Nature 321:522-525 (1986); Riechmann et al. Nature 332:323-327 (1988); and Presta Curr. Op. Struct. Biol. 2:593-596 (1992); which are incorporated herein by reference in its entirety. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and PCT International Publication No. WO 01/27160, where humanized antibodies and techniques for producing humanized antibodies having improved affinity for a predetermined antigen are disclosed.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen binding residues.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The antibody “tanezumab” is a humanized immunoglobulin G Type 2 (IgG2) monoclonal antibody directed against human nerve growth factor (NGF). Tanezumab binds to human NGF with high affinity and specificity and blocks the activity of NGF effectively in cell culture models. Tanezumab and/or its murine precursor have been shown to be an effective analgesic in animal models of pathological pain including arthritis, cancer pain, and post-surgical pain. Tanezumab has the sequences for the variable heavy chain region and variable light chain region of SEQ ID Nos: 1 and 2, respectively. The heavy chain and light chain sequences are provided in SEQ ID NOs: 9 and 10, or SEQ ID NOs: 11 and 10. The C-terminal lysine (K) of the heavy chain amino acid sequence of SEQ ID NO: 9 is optional and may be processed, resulting in a heavy chain amino acid sequence lacking the C-terminal lysine (K) and having the sequence shown in SEQ ID NO: 11. Sequences of tanezumab are provided in Table 1 below. Tanezumab is described, as antibody E3, in WO2004/058184, herein incorporated by reference.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a target (e.g., PD-1) epitope is an antibody that binds this epitope with greater affinity, avidity, more readily, and/or with greater duration than it binds to other target epitopes or non-target epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As used in the art, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

A “functional Fc region” possesses at least one effector function of a native sequence Fc region. Exemplary “effector functions” include C1q binding; complement dependent cytotoxicity; Fc receptor binding; antibody-dependent cell-mediated cytotoxicity; phagocytosis; down-regulation of cell surface receptors (e.g. B cell receptor), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably, from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably, at least about 90% sequence identity therewith, more preferably, at least about 95%, at least about 96%, at least about 97%, at least about 98%, at least about 99% sequence identity therewith.

As used herein, “treatment” is an approach for obtaining beneficial or desired clinical results. For purposes of this invention, beneficial or desired clinical results include reduction or improvement in signs and symptoms of osteoarthritis, for example as compared to before administration of the anti-NGF antibody.

“Ameliorating” means a lessening or improvement of one and more signs or symptoms of osteoarthritis, for example as compared to not administering an anti-NGF antibody as described herein. “Ameliorating” also includes shortening or reduction in duration of a symptom.

As used herein, an “effective dosage” or “effective amount” of drug, compound, or pharmaceutical composition is an amount sufficient to effect any one or more beneficial or desired results. In more specific aspects, an effective amount prevents, alleviates or ameliorates signs or symptoms of osteoarthritis, and/or prolongs the survival of the subject being treated. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing one or more signs or symptoms of osteoarthritis such as, for example, osteoarthritis of the hip, knee, shoulder or hand, decreasing the dose of other medications required to treat the disease, enhancing the effect of another medication, and/or delaying the progression of the disease in patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

The term “inadequate pain relief or intolerance to analgesic therapy” refers to a patient who has experienced an adverse event after treatment with the analgesic; who is refractory to treatment with the analgesic; who shows no clinically meaningful improvement in one or more measures of signs and symptoms of osteoarthritis including pain; who is addicted to the analgesic therapy (including opioids); or who is unwilling to take the analgesic therapy. Thus, the term includes reference to a patient for whom use of other analgesics is ineffective or not appropriate.

Treatment “effectively improves” or “effectively reduces” when assessment of the sign or symptom of osteoarthritis is quantified via a clinical measure relative to baseline and during and/or after the treatment period. The difference between the clinical measure at baseline and during/after treatment is compared and used to determine whether the sign or symptom has improved and the treatment is effective. This comparison can include comparison to placebo or to one or more of the prior therapies. In some embodiments, the comparison can be to placebo or to treatment with an analgesic therapy, such as an opioid or an NSAID. In some embodiments, the comparison can be to a sign or symptom before start of treatment with the NGF antibody. For example, the WOMAC Pain subscale measure can be determined for the patient at baseline and then determined throughout the treatment period, such as at weeks 2, 4, 8, 16, 24, 32, 40, 48, 56, or longer. Similarly, the WOMAC Physical Function subscale measure can also be determined in this manner. Yet further, the PGA-OA measure can also be determined in this manner.

In some embodiments the treatment effectively reduces WOMAC Pain by at least about 2.5, at least about 2.6, at least about 2.7, at least about 2.8, at least about 2.9, at least about 3.0, at least about 3.1, at least about 3.2, at least about 3.3, or at least about 3.4, at least about 3.5, at least about 3.6, at least about 3.7 compared to baseline WOMAC Pain prior to or at start of treatment. In some embodiments, the treatment reduces WOMAC Pain by greater than 20%^{, 25}%^{, 30}%^{, 35}%^{, 40}%^{, 45}%^{, 50}%^{, 55}%, 60%, 65%, 70%, 75%, 80%, 85%, 90% or 95% A compared to baseline prior to or at start of treatment. In some embodiments the treatment effectively reduces WOMAC Pain score compared to placebo for tanezumab. In some embodiments the treatment effectively reduces WOMAC Pain score by at least about 0.2, 0.25, 0.3, 0.35, 0.4, 0.45, 0.5, 0.55, 0.6, 0.65, 0.7, 0.75, 0.8, 0.85, 0.9, 0.95, or 1 compared to placebo for tanezumab. In some embodiments the treatment effectively reduces WOMAC Pain score compared to baseline and/or placebo for tanezumab to a greater extent than an opioid analgesic or an NSAID analgesic. In some embodiments the treatment reduces WOMAC Pain score by at least about 0.1, 0.15, 0.2, 0.25, 0.3, 0.35 more than an NSAID. In some embodiments the reduction in WOMAC Pain score is observed at week 16, 24, 32, 40, 48 or 56 of treatment. In some embodiments the change from baseline is based on the Least Squares Mean.

In some embodiments the treatment effectively reduces WOMAC Physical Function score by at least about 2.5, at least about 2.6, at least about 2.7, at least about 2.8, at least about 2.9, at least about 3.0, at least about 3.1, at least about 3.2, at least about 3.3, or at least about 3.4, at least about 3.5, at least about 3.6, at least about 3.7 compared to baseline prior to or at start of treatment. In some embodiments, the treatment reduces WOMAC Physical Function by greater than 20%, 25%, 30%, 35%, 40%, 45%, 50%, 55%, 60%, 65%, 70%, 75%, 80%, 85%, 90% or 95% compared to baseline prior to or at start of treatment. In some embodiments the treatment effectively reduces WOMAC Physical Function score compared to placebo for tanezumab. In some embodiments the treatment effectively reduces WOMAC Physical Function score by at least about 0.2, 0.25, 0.3, 0.35, 0.4, 0.45, 0.5, 0.55, 0.6, 0.65, 0.7, 0.75, 0.8, 0.85, 0.9, 0.95, 1 or 1.1 compared to placebo for tanezumab. In some embodiments the treatment effectively reduces WOMAC Physical Function score compared to baseline and/or placebo to a greater extent than an opioid analgesic or an NSAID analgesic. In some embodiments the treatment reduces WOMAC Physical Function score by at least about 0.1, 0.15, 0.2, 0.25, 0.3, 0.35, 0.4 more than an NSAID. In some embodiments the reduction in WOMAC Physical Function score is observed at week 16, 24, 32, 40, 48 or 56 of treatment. In some embodiments the change from baseline is based on the Least Squares Mean.

In some embodiments the treatment effectively reduces PGA-OA score by at least about 0.7, 0.75, 0.8, 0.85, 0.9, 0.95, 1, or 1.1 compared to baseline prior to or at start of treatment. In some embodiments, the treatment reduces PGA-OA by greater than 15%, 20%, 25%, 30% or 40% compared to baseline prior to or at start of treatment. In some embodiments the treatment effectively reduces PGA-OA score compared to placebo for tanezumab. In some embodiments the treatment effectively reduces PGA-OA score by at least about 0.1, 0.15, 0.2, 0.25, 0.3, 0.35, 0.4, 0.45, or 0.5 compared to placebo for tanezumab. In some embodiments the treatment effectively reduces PGA-OA score compared to baseline and/or placebo for tanezumab to a greater extent than an opioid analgesic or an NSAID analgesic. In some embodiments the reduction in PGA-OA score is observed at week 16, 24, 32, 40, 48 or 56 of treatment. In some embodiments the change from baseline is based on the Least Squares Mean.

The term “baseline” refers to a value of a sign or symptom associated measure for a patient prior to administration of the anti-NGF antibody as part of the treatment method. In some embodiments, the term “baseline” refers to a value of a sign or symptom associated measure for control healthy subjects that do not have osteoarthritis.

In some embodiments, treatment with the anti-NGF antibody effectively improves signs and symptoms of OA by at least 8 weeks after start of treatment with the antibody. In some embodiments, treatment with the anti-NGF antibody effectively improves signs and symptoms of OA by at least 10 weeks after start of treatment with the antibody. In some embodiments, treatment with the anti-NGF antibody effectively improves signs and symptoms of OA by at least 12 weeks after start of treatment with the antibody. In some embodiments, treatment with the anti-NGF antibody effectively improves signs and symptoms of OA by at least 14 weeks after start of treatment with the antibody. In some embodiments, treatment with the anti-NGF antibody effectively improves signs and symptoms of OA by at least 16 weeks after start of treatment with the antibody. In some embodiments, treatment with the anti-NGF antibody effectively improves signs and symptoms of OA by at least 24 weeks after start of treatment with the antibody. In some embodiments, treatment with the anti-NGF antibody effectively improves signs and symptoms of OA by at least 32 weeks after start of treatment with the antibody. In some embodiments, treatment with the anti-NGF antibody effectively improves signs and symptoms of OA by at least 40 weeks after start of treatment with the antibody.

In some embodiments, treatment with the anti-NGF antibody effectively improves signs and symptoms of OA within 1 week after start of treatment with the antibody. In some embodiments, treatment with the anti-NGF antibody effectively improves signs and symptoms of OA within 2 weeks after start of treatment with the antibody.

In some embodiments, the treatment improves pain associated with OA. In some embodiments the pain is moderate to severe pain associated with OA; and is optionally chronic pain.

The WOMAC Pain subscale is comprised of 5 questions regarding the amount of pain experienced due to OA in the index joint (selected study knee or hip) in the past 48 hours. The WOMAC Pain subscale is calculated as the mean of the scores from the five individual questions, which may not be a whole (integer) number. The WOMAC Pain subscale NRS scores for each question, and the WOMAC Pain subscale score, range from 0 to 10, with higher scores indicating higher pain.

The WOMAC Physical function subscale is comprised of 17 questions regarding the degree of difficulty experienced due to arthritis in the index joint (selected study knee or hip) in the past 48 hours. The WOMAC Physical Function subscale is calculated as the mean of the scores from the seventeen individual questions, which may not be a whole (integer) number. The WOMAC Physical Function subscale NRS scores for each question, and the WOMAC Physical Function subscale score, range from 0 to 10 with higher scores indicating worse function. This refers to the subject's ability to move around and perform usual activities of daily living.

The PGA-OA measure is based on a question to patients: “Considering all the ways your osteoarthritis in your [joint] affects you, how are you doing today?”. Patients rate their condition using the following scale:

Grade Description

1—Very Good—Asymptomatic and no limitation of normal activities

2—Good—Mild symptoms and no limitation of normal activities

3—Fair—Moderate symptoms and limitation of some normal activities

4—Poor—Severe symptoms and inability to carry out most normal activities

5—Very Poor—Very severe symptoms which are intolerable and inability to carry out all normal activities

Kellgren-Lawrence x-ray grade is a method of classifying the severity of osteoarthritis (Kellgren and Lawrence., Ann Rheum Dis 2000: 16(4): 494-502).

Rapidly progressive osteoarthritis (RPOA) of the hip was first described by Forestier in 1957 and subsequently described in a number of studies as atrophic osteoarthritis, rapidly destructive osteoarthritis, rapidly destructive arthropathy, rapidly progressive hip disease, or rapidly destructive coxarthrosis. Rapidly progressive hip osteoarthritis is characterized by subjects who typically present with hip pain, often severe, with radiographs that show rapid joint space narrowing as a result of chrondrolysis from a prior radiograph and, subsequently, an osteolytic phase with severe progressive atrophic bone destruction involving the femoral head and the acetabulum. There can be marked flattening of the femoral head and loss of subchondral bone in the weight bearing area and in some cases the femoral head appears sheared off. Osteophytes are typically conspicuously small or absent. Bone sclerosis is often present at sites of impaction of the femoral head and the acetabulum, subchondral detritus is invariably present and bone fragmentation and debris are commonly observed that can lead to synovitis. Lequesne proposed that subjects with 2 mm/year or greater of joint space narrowing or loss of more than 50% of the joint space within 1 year should be considered to have rapidly progressive osteoarthritis. Due to a lack of longitudinal studies, it is not clear what proportion of subjects with rapid loss of joint space (chondrolysis) will progress to have bone destruction. Rapid progression of osteoarthritis has also been described in the shoulder and the knee.

The incidence of rapidly progressive osteoarthritis in the overall osteoarthritis population is not well defined. For rapid progression of hip osteoarthritis, the prevalence ranges from approximately 2% to 18% based on clinical case series analyses. The pathophysiology of rapidly progressive osteoarthritis is not understood. Various mechanisms have been proposed including; ischemia, venous stasis, local nutritional deficiencies, synovitis, mechanical overloading, NSAID or corticosteroid use, intra articular deposition of hydroxyapatite or pyrophosphate crystals and subchondral insufficiency fractures.

There is a lack of data in the literature on the rate of rapidly progressive OA in a progressed OA population and the causes of this disease progression. As described by Hochberg et al (Arthritis Rheumatol., vol. 68, no. 2. pp. 382-391). “Rapidly progressive osteoarthritis is characterized by pain, with radiographs showing rapid joint space narrowing as a result of chondrolysis (type-1).” Possibly subsequently, these patients progress to an osteolytic phase with severe progressive atrophic bone destruction (type-2). However, this continuity is not clear due to a lack of longitudinal studies (Hochberg et al., Arthritis Rheumatol., vol. 68, no. 2. pp. 382-391).

The term “Index joint” refers to the most painful joint at screening before start of treatment and is the joint that is assessed during treatment. For example, the index joint is the most painful joint of the left and right hips and knees at screening before start of the treatment.

Radiographic assessments (x-rays) of both knees, both hips and both shoulders can be performed or obtained prior to treatment, at screening, and also during treatment. Other major joints exhibiting signs or symptoms suggestive of osteoarthritis may also be imaged. A major joint is defined as a mobile synovial joint in the limbs such as shoulders, elbows, wrists, hips, knees, ankles and excluding the joints of the toes and hands. Any joint imaged at screening or other at risk joints identified during the study period should also be imaged.

A central radiology reader (Central Reader) may review the radiology images for assessment of eligibility including determination and identification of exclusionary joint conditions. Radiographs required at screening may be obtained at least two weeks prior to the beginning of the Initial Pain Assessment Period (IPAP) to permit central radiology review of the images and to establish subject eligibility for initial dosing with an NGF antibody. In some embodiments, subjects may not be permitted to start dosing with an NGF antibody until the screening radiographs are reviewed and eligibility is established.

The X-ray technologists, in addition to their professional training and certifications, are trained in performing the radiographic protocols for the knees, hips, and shoulders. To facilitate reproducibility and accuracy of joint space width measurement in the knees and hips, a semi-automated software and positioning frame standardized subject and joint positioning protocol can be utilized. The Core Imaging Laboratory may be responsible for working with the sites to ensure quality, standardization and reproducibility of the radiographic images/assessments made at the Screening and follow-up time-points. Additional details regarding the required X-rays may be provided in a site imaging manual.

Central radiology readers (Central Readers) may be board certified radiologists or have the international equivalent as musculoskeletal radiologists. The Central Readers may be governed by an imaging atlas and an imaging Charter which includes a specific description of the scope of their responsibilities. Central Readers may review the radiology images at Screening for assessment of eligibility (including determination of Kellgren-Lawrence Grade) and identification of exclusionary joint conditions such as rapidly progressive osteoarthritis, atrophic or hypotrophic osteoarthritis, subchondral insufficiency fractures (spontaneous osteonecrosis of the knee [SPONK]), primary osteonecrosis and pathological fractures. After start of treatment, the Central Reader may review radiology images for diagnosis of joint conditions that would warrant further evaluation by the Adjudication Committee such as possible or probable rapidly progressive osteoarthritis, subchondral insufficiency fractures (spontaneous osteonecrosis of the knee [SPONK]), primary osteonecrosis or pathological fracture.

For subjects who are identified with a possible or probable joint event (i.e., rapidly progressive osteoarthritis, subchondral insufficiency fractures, spontaneous osteonecrosis of the knee (SPONK), primary osteonecrosis or pathological fracture) and subjects undergoing total joint replacement for any reason, all images and other source documentation may be provided to the blinded Adjudication Committee for review and adjudication of the event. The Adjudication Committee's assessment of the event may represent the final classification of the event.

Patients may be excluded from treatment with the anti-NGF antibody, during or before treatment with the anti-NGF antibody, if there is radiographic evidence of any of the following conditions in any screening radiograph as determined by a central radiology reviewer and as defined in an imaging atlas: excessive malalignment of the knee, severe chondrocalcinosis; other arthropathies (e.g., rheumatoid arthritis), systemic metabolic bone disease (e.g., pseudogout, Paget's disease; metastatic calcifications), large cystic lesions, primary or metastatic tumor lesions, stress or traumatic fracture. In some embodiments a patient may be excluded from treatment with the anti-NGF antibody, before or during the treatment with the anti-NGF antibody, if there is radiographic evidence of any of the following conditions as determined by the central radiology reviewer and as defined in an imaging atlas at screening: 1) rapidly progressive osteoarthritis, 2) atrophic or hypotrophic osteoarthritis, 3) subchondral insufficiency fractures, 4) spontaneous osteonecrosis of the knee (SPONK), 5) osteonecrosis, or 6) pathologic fracture.

In some embodiments the patents are monitored for the development of signs and symptoms of rapidly progressive osteoarthritis prior to each dose. In some embodiments monitoring include radiographic assessment (such as X-ray). In some embodiments the radiographic assement is performed annually during treatment. The radiographic assessment may be a bilateral assessment of the hip and/or knee. In some embodiments, symptoms of rapidly progressive osteoarthritis may include new onset, severe persistent pain or swelling in a joint. In some embodiments, treatment is discontinued if a patient develops rapidly progressive osteoarthritis.

A “patient”, an “individual” or a “subject”, used interchangeably herein, is a mammal, more preferably, a human. Mammals also include, but are not limited to, farm animals (e.g., cows, pigs, horses, chickens, etc.), sport animals, pets, primates, horses, dogs, cats, mice and rats.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

The term “effector function” refers to the biological activities attributable to the Fc region of an antibody. Examples of antibody effector functions include, but are not limited to, antibody-dependent cell-mediated cytotoxicity (ADCC), Fc receptor binding, complement dependent cytotoxicity (CDC), phagocytosis, C1q binding, and down regulation of cell surface receptors (e.g., B cell receptor; BCR). See, e.g., U.S. Pat. No. 6,737,056. Such effector functions generally require the Fc region to be combined with a binding domain (e.g., an antibody variable domain) and can be assessed using various assays known in the art for evaluating such antibody effector functions. An exemplary measurement of effector function is through Fcγ3 and/or C1q binding.

As used herein “antibody-dependent cell-mediated cytotoxicity” or “ADCC” refers to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al., 1998, PNAS (USA), 95:652-656.

“Complement dependent cytotoxicity” or “CDC” refers to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202: 163 (1996), may be performed.

The term “kₒₙ” or “kₐ”, as used herein, refers to the rate constant for association of an antibody to an antigen. Specifically, the rate constants (kₒₙ or kₐ and k_{off} or k_{d}) and equilibrium dissociation constants are measured using whole antibody (i.e. bivalent) and monomeric proteins.

The term “k_{off}” or “k_{d}”, as used herein, refers to the rate constant for dissociation of an antibody from the antibody/antigen complex.

The term “K_{D}”, as used herein, refers to the equilibrium dissociation constant of an antibody-antigen interaction.

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

The term “subcutaneous administration” refers to the administration of a substance into the subcutaneous layer.

The term “preventing” or “prevent” refers to (a) keeping a disorder from occurring or (b) delaying the onset of a disorder or onset of symptoms of a disorder.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present invention. The materials, methods, and examples are illustrative only and not intended to be limiting.

### Anti-NGF Antibodies

Provided herein are anti-NGF antibodies for use in the method of treatment.

In one aspect, the anti-NGF antibody binds to NGF and inhibits binding of NGF to trkA and/or p75.

In an embodiment, the antibody comprises three CDRs from the heavy chain variable region of SEQ ID NO: 1. In some embodiments, the antibody comprises three CDRs from the light chain variable region of SEQ ID NO: 2. In some embodiments the antibody comprises three CDRs from the heavy chain variable region of SEQ ID NO: 1 and three CDRs from the light chain variable region of SEQ ID NO: 2.

In some embodiments, the CDRs may be defined in accordance with any of Kabat, Chothia, extended, AbM, contact, and/or conformational definitions. In some embodiments, the CDRS shown in SEQ ID NO:3, SEQ ID NO:4, SEQ ID NO:5, SEQ ID NO:6, SEQ ID NO:7, and SEQ ID NO:8 are determined by a combination of the Kabat and Chothia methods.

Exemplary antibody sequences used for the present invention include, but are not limited to, the sequences listed below.

TABLE 1

| SEQ | |
|---|---|
| ID | |
| NO: | Sequence |
| 1 | Variable heavy chain region: |
| | QVQLQESGPGLVKPSETLSLTCTVSGFSLIGYDLNWIRQPPGKGL |
| | EWIGIIWGDGTTDYNSAVKSRVTISKDTSKNQFSLKLSSVTAADT |
| | AVYYCARGGYWYATSYYFDYWGQGTLVTVS |
| 2 | Variable light chain region: |
| | DIQMTQSPSSLSASVGDRVTITCRASQSISNNLNWYQQKPGKAPK |
| | LLIYYTSRFHSGVPSRFSGSGSGTDFTFTISSLQPEDIATYYCQQ |
| | EHTLPYTFGQGTKLEIKRT |
| 3 | Extended HCDR1: |
| | GFSLIGYDLN |
| 4 | Extended HCDR2: |
| | IIWGDGTTDYNSAVKS |
| 5 | Extended HCDR3: |
| | GGYWYATSYYFDY |
| 6 | Extended LCDR1: |
| | RASQSISNNLN |
| 7 | Extended LCDR2: |
| | YTSRFHS |
| 8 | Extended LCDR3: |
| | QQEHTLPYT |
| 9 | Heavy chain*: |
| | QVQLQESGPGLVKPSETLSLTCTVSGFSLIGYDLNWIRQPPGKGL |
| | EWIGIIWGDGTTDYNSAVKSRVTISKDTSKNQFSLKLSSVTAADT |
| | AVYYCARGGYVVYATSYYFDYWGQGTLVTVSSASTKGPSVFPLAP |
| | CSRSTSESTAALGCLVKDYFPEPVTVSWNSGALTSGVHTFPAVLQ |
| | SSGLYSLSSVVTVPSSNFGTQTYTCNVDHKPSNTKVDKTVERKCC |
| | VECPPCPAPPVAGPSVFLFPPKPKDTLMISRTPEVTCVVVDVSHE |
| | DPEVQFNWYVDGVEVHNAKTKPREEQFNSTFRVVSVLTVVHQDWL |
| | NGKEYKCKVSNKGLPSSIEKTISKTKGQPREPQVYTLPPSREEMT |
| | KNQVSLTCLVKGFYPSDIAVEWESNGQPENNYKTTPPMLDSDGSF |
| | FLYSKLTVDKSRWQQGNVFSCSVMHEALHNHYTQKSLSLSPGK |
| 10 | Light chain: |
| | DIQMTQSPSSLSASVGDRVTITCRASQSISNNLNVVYQQKPGKAP |
| | KLLIYYTSRFHSGVPSRFSGSGSGTDFTFTISSLQPEDIATYYCQ |
| | QEHTLPYTFGQGTKLEIKRTVAAPSVFIFPPSDEQLKSGTASVVC |
| | LLNNFYPREAKVQWKVDNALQSGNSQESVTEQDSKDSTYSLSSTL |
| | TLSKADYEKHKVYACEVTHQGLSSPVTKSFNRGEC |
| 11 | Heavy chain (C-terminal lysine (K) processed) |
| | QVQLQESGPGLVKPSETLSLTCTVSGFSLIGYDLNWIRQPPGKGL |
| | EWIGIIWGDGTTDYNSAVKSRVTISKDTSKNQFSLKLSSVTAADT |
| | AVYYCARGGYVVYATSYYFDYWGQGTLVTVSSASTKGPSVFPLAP |
| | CSRSTSESTAALGCLVKDYFPEPVTVSWNSGALTSGVHTFPAVLQ |
| | SSGLYSLSSVVTVPSSNFGTQTYTCNVDHKPSNTKVDKTVERKCC |
| | VECPPCPAPPVAGPSVFLFPPKPKDTLMISRTPEVTCVVVDVSHE |
| | DPEVQFNWYVDGVEVHNAKTKPREEQFNSTFRVVSVLTVVHQDWL |
| | NGKEYKCKVSNKGLPSSIEKTISKTKGQPREPQVYTLPPSREEMT |
| | KNQVSLTCLVKGFYPSDIAVEWESNGQPENNYKTTPPMLDSDGSF |
| | FLYSKLTVDKSRWQQGNVFSCSVMHEALHNHYTQKSLSLSPG |

[*C-terminal lysine (K) of the heavy chain amino acid sequence of SEQ ID NO: 9 is optional]

In one embodiment, the antibody is tanezumab.

In some embodiments, the antibody comprises a HCDR1 having the sequence shown in SEQ ID NO:3, a HCDR2 having the sequence shown in SEQ ID NO:4, a HCDR3 having the sequence shown in SEQ ID NO:5, a LCDR1 having the sequence shown in SEQ ID NO:6, a LCDR2 having the sequence shown in SEQ ID NO:7, and a LCDR3 having the sequence shown in SEQ ID NO:8.

In some embodiments, the antibody comprises a heavy chain variable region (VH) having the sequence shown in SEQ ID NO: 1. In some embodiments, the antibody comprises a light chain variable region (VL) having the amino acid sequence of SEQ ID NO: 2. In some embodiments, the antibody comprises a heavy chain variable region (VH) having the sequence shown in SEQ ID NO: 1 and a light chain variable region (VL) having the amino acid sequence of SEQ ID NO: 2.

In some embodiments, the antibody comprises a heavy chain having the amino acid sequence shown in SEQ ID NO: 9 and a light chain having the amino acid sequence shown in SEQ ID NO: 10. In some embodiments, the C-terminal lysine (K) of the heavy chain amino acid sequence of SEQ ID NO: 9 is optional. Thus, in some embodiments the heavy chain amino acid sequence lacks the C-terminal lysine (K) and has the sequence shown in SEQ ID NO: 11. Thus, in some embodiments, the antibody comprises a heavy chain having the amino acid sequence shown in SEQ ID NO: 11 and a light chain having the amino acid sequence shown in SEQ ID NO: 10.

In some embodiments, the antibody is fasinumab or REGN475 (see, for example, US 2009/0041717, herein incorporated by reference) or has the same or substantially the same amino acid sequence as fasinumab or REGN475. In some embodiments, the antibody is fulranumab.

The antibodies as described herein can be made by any method known in the art. An antibody may be made recombinantly using a suitable host cell. A nucleic acid encoding an anti-NGF antibody of the present disclosure can be cloned into an expression vector, which can then be introduced into a host cell, where the cell does not otherwise produce an immunoglobulin protein, to obtain the synthesis of an antibody in the recombinant host cell. Any host cell susceptible to cell culture, and to expression of protein or polypeptides, may be utilized in accordance with the present invention. In certain embodiments, the host cell is mammalian. Mammalian cell lines available as hosts for expression are well known in the art and include many immortalized cell lines available from the American Type Culture Collection (ATCC). Nonlimiting exemplary mammalian cells include, but are not limited to, NS0 cells, HEK 293 and Chinese hamster ovary (CHO) cells, and their derivatives, such as 293-6E and CHO DG44 cells, CHO DXB11, and Potelligent® CHOK1SV cells (BioWa/Lonza, Allendale, N.J.). Mammalian host cells also include, but are not limited to, human cervical carcinoma cells (HeLa, ATCC CCL 2), baby hamster kidney (BHK, ATCC CCL 10) cells, monkey kidney cells (COS), and human hepatocellular carcinoma cells (e.g., Hep G2). Other non-limiting examples of mammalian cells that may be used in accordance with the present invention include human retinoblasts (PER.C6®; CruCell, Leiden, The Netherlands); monkey kidney CV1 line transformed by SV40 (COS-7, ATCC CRL 1651); human embryonic kidney line 293 (HEK 293) or 293 cells subcloned for growth in suspension culture (Graham et al., J. Gen Virol. 1997; 36:59); mouse sertoli cells (TM4, Mather, Biol. Reprod. 1980; 23:243-251); monkey kidney cells (CV1 ATCC CCL 70); African green monkey kidney cells (VERO-76, ATCC CRL-1 587); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 1982; 383:44-68); MRC 5 cells; FS4 cells; a human hepatoma line (Hep G2); and numerous myeloma cell lines, including, but not limited to, BALB/c mouse myeloma line (NS0/1, ECACC No: 85110503), NS0 cells and Sp2/0 cells.

Additionally, any number of commercially and non-commercially available cell lines that express polypeptides or proteins may be utilized. One skilled in the art will appreciate that different cell lines might have different nutrition requirements and/or might require different culture conditions for optimal growth and polypeptide or protein expression and will be able to modify conditions as needed.

For the production of hybridoma cell lines, the route and schedule of immunization of the host animal are generally in keeping with established and conventional techniques for antibody stimulation and production, as further described herein. General techniques for production of human and mouse antibodies are known in the art and/or are described herein.

It is contemplated that any mammalian subject including humans or antibody producing cells therefrom can be manipulated to serve as the basis for production of mammalian, including human and hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C., Nature 256:495-497, 1975 or as modified by Buck, D. W., et al., In Vitro, 18:377-381, 1982. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies.

Hybridomas that produce antibodies used for the present invention may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen. Immunization of a host animal with cells expressing the antibody target (e.g., PD-1), a human target protein (e.g., PD-1), or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups, can yield a population of antibodies (e.g., monoclonal antibodies).

If desired, the antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., J. Immunol. Methods 329, 112, 2008; U.S. Pat. No. 7,314,622.

In some embodiments, antibodies may be made using hybridoma technology. It is contemplated that any mammalian subject including humans or antibody producing cells therefrom can be manipulated to serve as the basis for production of mammalian, including human, hybridoma cell lines. The route and schedule of immunization of the host animal are generally in keeping with established and conventional techniques for antibody stimulation and production, as further described herein. Typically, the host animal is inoculated intraperitoneally, intramuscularly, orally, subcutaneously, intraplantar, and/or intradermally with an amount of immunogen, including as described herein.

In some embodiments, antibodies as described herein are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

Glycosylation of antibodies is typically either N-linked or O-linked. N-linked refers to the attachment of the carbohydrate moiety to the side chain of an asparagine residue. The tri peptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

### Polynucleotides, Vectors, and Host Cells

The invention also provides polynucleotides encoding any of the anti-NGF antibodies as described herein. In one aspect, the invention provides a method of making any of the polynucleotides described herein. Polynucleotides can be made and expressed by procedures known in the art.

In another aspect, the invention provides compositions (such as a pharmaceutical compositions) comprising any of the polynucleotides of the invention. In some embodiments, the composition comprises an expression vector comprising a polynucleotide encoding any of the anti-NGF antibodies described herein.

In another aspect, provided is an isolated cell line that produces the anti-NGF antibodies as described herein.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an antibody or a fragment thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a fragment thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene® suite of bioinformatics software (DNASTAR®, Inc., Madison, Wis.), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P.H.A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention.

Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). Preferably, the host cells express the cDNAs at a level of about 5 fold higher, more preferably, 10 fold higher, even more preferably, 20 fold higher than that of the corresponding endogenous antibody or protein of interest, if present, in the host cells. Screening the host cells for a specific binding to NGF is effected by an immunoassay or FACS. A cell overexpressing the antibody or protein of interest can be identified.

### Compositions

The invention also provides pharmaceutical compositions comprising an effective amount of an anti-NGF antibody as described herein. Examples of such compositions, as well as how to formulate, are also described herein.

It is understood that the compositions can comprise more than one anti-NGF antibody.

The composition used in the present invention can further comprise pharmaceutically acceptable carriers, excipients, or stabilizers (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

The anti-NGF antibody, and compositions thereof, can also be used in conjunction with, or administered separately, simultaneously, or sequentially with other agents that serve to enhance and/or complement the effectiveness of the agents.

### Methods for Improving Signs and Symptoms of Osteoarthritis

In one aspect, the invention provides a method for treating signs and symptoms of osteoarthritis (OA) in a patient.

In some embodiments, the methods described herein further comprise a step of treating a subject with an additional form of therapy. In some embodiments, the additional form of therapy is an additional therapeutic agent which may be selected from an NGF antagonist, a trkA antagonist, an IL-1 antagonist, an IL-6 antagonist, an IL-6R antagonist, an opioid, acetaminophen, a local anesthetic, an NMDA modulator, a cannabinoid receptor agonist, a P2X family modulator, a VR1 antagonist, a substance P antagonist, a Nav1.7 antagonist, a cytokine or cytokine receptor antagonist, a steroid, other inflammatory inhibitors and a corticosteroid.

In some embodiments, the method described herein does not comprise administration of an NSAID to the patient. In some embodiments, the method described herein does not comprise administration of an opioid to the patient.

With respect to all methods described herein, reference to anti-NGF antibodies also includes compositions comprising one or more additional agents. These compositions may further comprise suitable excipients, such as pharmaceutically acceptable excipients including buffers, which are well known in the art. The present invention can be used alone or in combination with other methods of treatment.

The anti-NGF antibodies as described herein are administered to a subject via systemic administration (e.g., intravenous or subcutaneous administration). Preferably the antibodies are administered via subcutaneous injection.

Various formulations of an anti-NGF antibody may be used for administration. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing, 2000.

In some embodiments, these agents are formulated for administration by injection (e.g., intraperitoneally, intravenously, subcutaneously, intramuscularly, intraarticularly etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

In some embodiments the anti-NGF antibody, such as tanezumab, is administered in a formulation described in WO2010/032220, herein incorporated by reference.

In some embodiments, the formulation is a liquid formulation and comprises an anti-NGF antibody at a concentration of about 2.5 mg/ml, 5 mg/ml, 10 mg/ml or 20 mg/ml; and a histidine buffer.

In some embodiments, the formulation further comprises a surfactant which may be polysorbate 20. In some embodiments, the formulation further comprises trehalose dehydrate or sucrose. In some embodiments, the formulation further comprises a chelating agent, which may be EDTA; in some embodiments disodium EDTA. In some embodiments, the formulation is of pH 6.0±0.3.

In some embodiments, the formulation comprises about 2.5 mg/ml, 5 mg/ml, 10 mg/ml or 20 mg/ml tanezumab; about 10 mM histidine buffer; about 84 mg/ml trehalose dehydrate; about 0.1 mg/ml Polysorbate 20; about 0.05 mg/ml disodium EDTA; wherein the formulation is of a pH 6.0±0.3.

In some embodiments the formulation comprises about 2.5 mg/ml or 5 mg/ml. In some embodiments, the formulation has a total volume of about 1 ml.

In some embodiments the formulation is contained in a glass or plastic vial or syringe. In some embodiments the formulation is contained in a pre-filled glass or plastic vial or syringe.

The anti-NGF antibody can be administered every eight weeks. For repeated administrations over several doses, the treatment is sustained until a desired suppression of signs and symptoms of osteoarthritis occurs. The progress of this therapy can be monitored by conventional techniques and assays.

The dosing regimen (including the specific anti-NGF antibodies used) can vary over time. For example in some embodiments, the dosage is 2.5 mg administered every eight weeks. In some embodiments the dosage is 5 mg administered every eight weeks. In some embodiments the dosage of 2.5 mg can be increased to 5 mg for subsequent administrations. For example, the dosage of 2.5 mg can be administered at start of therapy and then a dosage of 5 mg can be administered at eight weeks, with a dosage of 5 mg being administered at sixteen weeks and each subsequent eight weekly dosage. In addition as another example, the dosage of 2.5 mg can be administered at start of therapy and at eight weeks, with a dosage of 5 mg being administered at sixteen weeks and each subsequent eight weekly dosage. In addition as another example, the 2.5 mg dosage can be administered at start of therapy and then for one, two, or more eight weekly dosages before subsequent dosages of 5 mg every eight weeks are administered.

In some aspects in which the antibody is fasinumab (see, for example, US 2009/0041717, herein incorporated by reference), the antibody is administered at a dose of between 0.5 mg to 50 mg. In some embodiments the antibody is administered at dose between 0.5 mg and 12 mg. In some embodiments the antibody is administered at a dose of 1 mg, 2 mg, 3 mg, 4 mg, 5 mg, 6 mg, 7 mg, 8 mg, 9 mg or 10 mg. In some embodiments the antibody is administered subcutaneously or intravenously. In some embodiments the antibody is administered every four weeks or every eight weeks.

In some aspects in which the antibody is comprises the same or substantially the same amino acid sequence as fasinumab (see, for example, US 2009/0041717, herein incorporated by reference), the antibody is administered at a dose of between 0.5 mg to 50 mg. In some embodiments the antibody is administered at dose between 0.5 mg and 12 mg. In some embodiments the antibody is administered at a dose of 1 mg, 2 mg, 3 mg, 4 mg, 5 mg, 6 mg, 7 mg, 8 mg, 9 mg or 10 mg. In some embodiments the antibody is administered subcutaneously or intravenously. In some embodiments the antibody is administered every four weeks or every eight weeks.

In some embodiments a loading dose (or induction dose) is administered followed by the administration of maintenance doses at a lower amount or at lower frequency.

For the purpose of the present invention, the appropriate dosage of an anti-NGF antibody will depend on the antibody employed, the type and severity of symptoms to be treated, whether the agent is administered for preventive or therapeutic purposes, previous therapy, the patient's clinical history and response to the agent, the patient's clearance rate for the administered agent, and the discretion of the attending physician. Typically the clinician will administer an anti-NGF antibody until a dosage is reached that achieves the desired result. Dose and/or frequency can vary over course of treatment. Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of symptoms.

In one embodiment, dosages for an anti-NGF antibody may be determined empirically in individuals who have been given one or more administration(s) of an anti-NGF antibody. For example, individuals are given incremental dosages of an anti-NGF antibody. To assess efficacy, an indicator of the signs and symptoms of osteoarthritis can be followed.

Administration of an anti-NGF antibody as described herein in accordance with the method in the present invention can be continuous or intermittent, depending, for example, upon the recipient's physiological condition, whether the purpose of the administration is therapeutic or prophylactic, and other factors known to skilled practitioners. The administration of an anti-NGF antibody may be essentially continuous over a preselected period of time or may be in a series of spaced doses.

In some embodiments, more than anti-NGF antibody may be present. At least one, at least two, at least three, at least four, at least five different, or more anti-NGF antibodies can be present. Generally, those anti-NGF antibodies may have complementary activities that do not adversely affect each other.

In some embodiments, the anti-NGF antibody may be administered in combination with the administration of one or more additional therapeutic agents.

In some embodiments, an anti-NGF antibody administration is combined with a treatment regimen further comprising a traditional therapy including surgery.

### Formulations

Therapeutic formulations of the anti-NGF antibody used in accordance with the present invention are prepared for storage by mixing the protein having the desired degree of purity with optional pharmaceutically acceptable carriers, excipients or stabilizers (Remington, The Science and Practice of Pharmacy 20th Ed. Mack Publishing, 2000), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Liposomes containing the anti-NGF antibody are prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for in vivo administration must be sterile. This is readily accomplished by, for example, filtration through sterile filtration membranes. Therapeutic anti-NGF antibody compositions are generally placed into a container having a sterile access port, for example, an intravenous solution bag or vial having a stopper pierceable by a hypodermic injection needle.

The compositions according to the present invention may be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from about 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an anti-NGF antibody with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

### Kits

The invention also provides kits comprising any or all of the anti-NGF antibodies described herein. Kits of the invention include one or more containers comprising an anti-NGF antibody described herein and instructions for use in accordance with any of the methods of the invention described herein. Generally, these instructions comprise a description of administration of the anti-NGF antibody for the above described therapeutic treatments. In some embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

The instructions relating to the use of an anti-NGF antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-NGF antibody. The container may further comprise a second pharmaceutically active agent.

Kits may optionally provide additional components such as buffers and interpretive information. Normally, the kit comprises a container and a label or package insert(s) on or associated with the container.

### EXAMPLES

### Example 1

Study Design

This was a randomized, double-blind, placebo-controlled, multicenter, parallel-group, dose-titration study (16-week treatment period, 24-week safety follow-up period) assessing the efficacy and safety of subcutaneous (SC) tanezumab in patients with moderate to severe OA of the hip or knee. All patients provided written informed consent before participating. The study was conducted in compliance with the Declaration of Helsinki and all International Conference on Harmonisation Good Clinical Practice guidelines.

The primary objective was to demonstrate superior efficacy of 2 SC tanezumab treatment regimens at week 16—fixed dosing (2.5 mg administered at baseline and week 8) and forced dose titration (2.5 mg administered at baseline and 5 mg at week 8)—compared with placebo treatment. The secondary objectives evaluated: (1) the efficacy of tanezumab titrated from 2.5 mg to 5 mg at week 8 compared with two administrations of tanezumab 2.5 mg 8 weeks apart; and (2) the safety of both tanezumab dosing regimens.

The study was divided into 3 periods: screening (≤37 days), treatment (16 weeks), and safety follow-up (24 weeks). Screening procedures included a washout period of prohibited medications and an Initial Pain Assessment Period (IPAP) (3-7 days prior to randomization/baseline).

Study Population

Patients were aged years and diagnosed with hip or knee OA according to American College of Rheumatology criteria with x-ray confirmation at screening (Kellgren-Lawrence x-ray grade Entry criteria included an index joint WOMAC Pain subscale at both screening and baseline, a baseline WOMAC Physical Function subscale and a baseline Patient's Global Assessment of Osteoarthritis (PGA-OA) of “fair,” “poor,” or “very poor.” Index joint was defined as the most painful hip or knee at screening. Patients had a documented history of: (1) insufficient pain relief from acetaminophen; (2) insufficient pain relief, intolerance to, or contraindication to NSAIDs; and (3) insufficient pain relief, intolerance to, or contraindication to either tramadol or opioids (or were unwilling to take opioids).

Main exclusion criteria included: evidence of prespecified joint safety conditions (eg, rapidly progressive OA [RPOA], subchondral insufficiency fracture, osteonecrosis, pathologic fracture) in any major joint on screening x-rays as determined by the central reader; a history of diseases that could confound index joint efficacy assessments (eg, rheumatoid arthritis, seronegative spondyloarthropathies, gout, chondrocalcinosis/pseudogout); significant trauma or surgery in a hip, knee, or shoulder in the previous year; any planned surgery during the study; any recent intra-articular corticosteroid or hyaluronic acid injection in the index joint; a history of neurological conditions (eg, peripheral or autonomic neuropathy, Alzheimer's disease, multiple sclerosis); a Survey of Autonomic Symptoms (Zilliox et al., 2011; 76(12):1099-1105) score>7 at screening; and pregnancy or breastfeeding.

Treatment

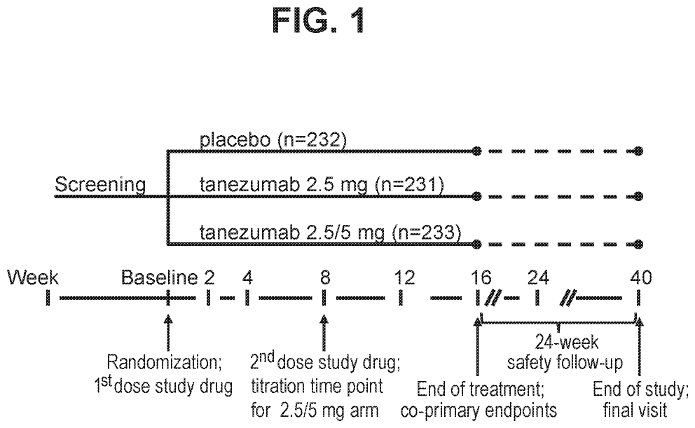

Using a computer-generated randomization code, patients were randomized in equal allocation to 1 of 3 SC treatment regimens: placebo at baseline and week 8 (i.e., placebo); tanezumab 2.5 mg at baseline and week 8 (i.e., tanezumab 2.5 mg); and tanezumab 2.5 mg at baseline and 5 mg at week 8 (i.e., tanezumab 2.5/5 mg; FIG. 1).

Permitted concomitant treatments included aspirin ≤325 mg/d for cardiovascular prophylaxis and stable doses of medications for other non-OA, non-pain conditions. Analgesics were prohibited except as follows. NSAIDs for non-OA conditions were permitted for up to 10 days/8-week period between baseline and week 24, but not within 48 hours of a study visit. Rescue medication (acetaminophen) was allowed ≤3000 mg/d and ≤3 days/week during the treatment period, but not within 24 hours of an in-clinic visit where efficacy assessments were collected. Standard of care treatment for OA pain was permitted 16 weeks after the last study drug dose.

Efficacy Assessments

The co-primary efficacy endpoints were change from baseline to week 16 in WOMAC Pain subscale, WOMAC Physical Function subscale, and PGA-OA (Theiler et al., Osteoarthritis Cartilage. 2002; 10(6):479-481; Stengaard-Pederson et al., Rheumatology (Oxford). 2004; 43(5):592-595). WOMAC Pain and WOMAC Physical Function subscales measured symptoms within the last 48 hours; both used an 11-point numeric rating scale where higher scores indicated higher levels of pain or worse function. For PGA-OA, patients answered the question “Considering all the ways your osteoarthritis in your hip/knee affects you, how are you doing today?” on a scale from 1=“very good” to 5=“very poor.”

A key secondary efficacy endpoint was the WOMAC Pain ≤50%, responder rate at week 16. Other secondary efficacy endpoints were WOMAC Pain responder rates (≥30%, ≤70%, and ≤90%) at week 16 and patient-reported rescue medication use during weeks 2, 4, 8, 12, and 16.

Individual patient transitions in WOMAC Pain response categories (non-response defined as <30% reduction in WOMAC Pain; moderate response as ≥30%, but <50% reduction in WOMAC Pain; and substantial response as ≤50%, reduction from baseline in WOMAC Pain) between adjacent time points through the study (from week 4 to week 8, from week 8 to week 12, and from week 12 to week 16), were investigated using contingency tables. These analyses were conducted post hoc. Consistent non-responders were those patients with less than a 30% reduction from baseline in WOMAC Pain at two consecutive time points. Consistent moderate responders were those patients with at least a 30% but less than 50% reduction from baseline in WOMAC Pain at two consecutive time points. Consistent substantial responders were those patients who maintained at least a 50% reduction from baseline in WOMAC Pain at two consecutive time points.

Safety Assessments

Safety assessments included: adverse event (AE) reporting; laboratory testing; 12-lead electrocardiogram; sitting vital signs; orthostatic blood pressure assessments; physical examinations; musculoskeletal examinations; neurological examinations reported using the Neuropathy Impairment Score (Dyck et al., Neurology. 1995; 45(6):1115-1121); Survey of Autonomic Symptoms scores (Zilliox et al., Neurology. 2011; 76(12):1099-1105); adjudication of joint safety events including total joint replacements (TJRs); and anti-drug antibody assessments. AEs were coded using MedDRA v21.0. AE severity and relationship to study treatment were assessed by the investigator.

Neurological Assessments

Patients meeting protocol-specified criteria were further evaluated for peripheral neuropathy and/or sympathetic autonomic neuropathy. Neurological examinations were conducted at all clinic visits by investigators or designated physicians who received protocol-required training, and findings were recorded using the Neuropathy Impairment Score (Dyck et al). Patients were referred to a consulting neurologist if an AE of peripheral neuropathy or abnormal peripheral sensation was reported as: (1) serious; (2) of severe intensity; (3) resulted in study withdrawal; or (4) was ongoing at the end of study participation. Patients with reported AEs suggestive of sympathetic autonomic neuropathy (i.e., bradycardia, orthostatic hypotension, syncope, anhidrosis, or hypohydrosis) of any seriousness or severity were further evaluated by a cardiologist or neurologist.

Joint Safety Events

Investigators performed musculoskeletal examinations at each study visit. Investigators evaluated patients reporting increased severe or persistent pain via eDiary lasting ≤2 weeks to determine if additional follow-up was needed. Post-baseline x-rays (scheduled or for cause) were assessed by the central reader for possible or probable RPOA, subchondral insufficiency fracture, primary osteonecrosis, or pathologic fracture. If warranted, magnetic resonance imaging scans were performed and/or patients were referred to an orthopedic surgeon. All cases of possible or probable joint safety events or cases of TJR for any reason were adjudicated by a blinded Adjudication Committee consisting of orthopedic surgery, rheumatology, orthopedic pathology, and musculoskeletal radiology experts.

Statistics

Using a combined analysis of 2 previous studies (Brown et al., J Pain. 2012; 13(8):790-798; Brown et al., Arthritis Rheum. 2013; 65(7):1795-1803), a sample size of approximately 230 subjects per treatment group was determined to provide 90% power to achieve statistical significance at the 5% 2-sided level for comparisons of tanezumab 2.5 mg and tanezumab 2.5/5 mg versus placebo across all 3 co-primary endpoints. Co-primary endpoints were analyzed in the intent-to-treat population (all patients who received ≥1 study medication dose) using an analysis of covariance model, with terms for baseline score, baseline patient diary average pain, index joint, Kellgren-Lawrence grade, and treatment group, and study site as a random effect. Missing data were handled with a multiple imputation strategy dependent on the reason for discontinuation.

The co-primary endpoints used a step-down strategy, defined as first testing tanezumab 2.5/5 mg versus placebo and, if statistically significant, then testing tanezumab 2.5 mg versus placebo. Tanezumab treatment groups were declared superior to placebo if all 3 co-primary endpoints were significant. The key secondary efficacy endpoint was tested using the Hochberg procedure for both tanezumab regimens versus placebo, contingent on successful primary comparisons. Comparisons for other secondary endpoints were unadjusted. Comparisons between the tanezumab dose regimens were descriptive only. Safety assessments were summarized by treatment group as percentages of the treatment group population.

Results

Patients

Randomization included 698 patients, and 696 received study treatment dose (2 randomized patients who did not meet eligibility criteria were discontinued prior to dosing). All patients who received study treatment dose were analyzed for efficacy and safety. Patient baseline characteristics were similar across treatment groups (Table 2).

Efficacy

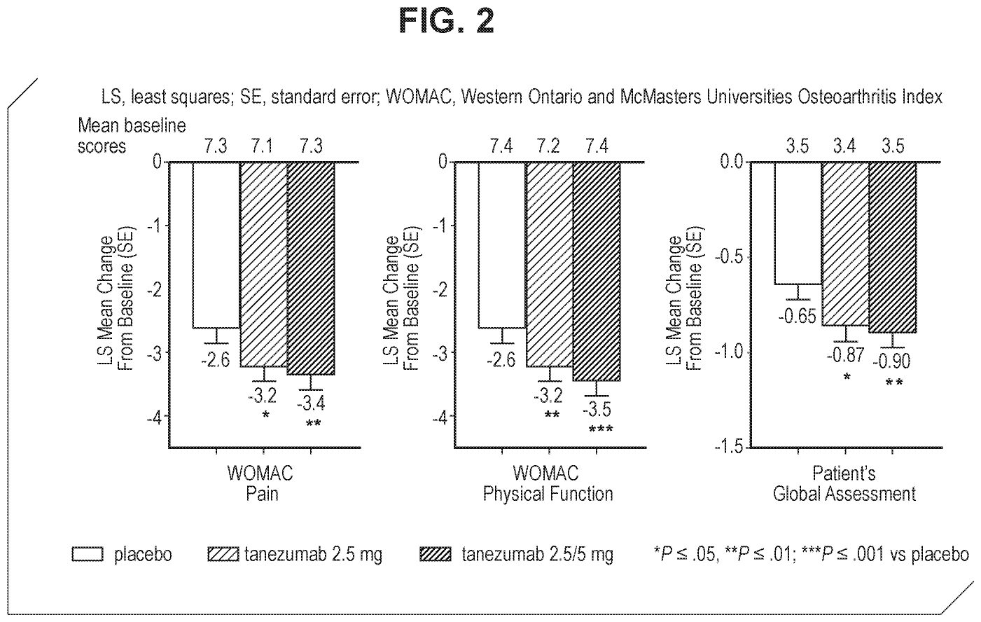

Both tanezumab 2.5 mg and 2.5/5 mg demonstrated statistically significant improvement in WOMAC Pain, WOMAC Physical Function, and PGA-OA compared with placebo at week 16 (P≤0.05; FIG. 2); thus, both tanezumab dosing regimens met the co-primary endpoints.

Tanezumab 2.5 mg (which both active treatment groups received at baseline) demonstrated efficacy within the first week. Compared with placebo, both tanezumab groups had statistically significant improvements in average daily index joint pain at week 1 (least squares [LS] mean difference±standard error [SE] versus placebo: −0.33±0.15 for tanezumab 2.5 mg group and −0.38±0.15 for tanezumab 2.5/5 mg group, both P<0.05), with onset evident on day 3 (tanezumab 2.5 mg group) and day 5 (tanezumab 2.5/5 mg group). Both tanezumab dosing regimens demonstrated statistically significant efficacy in WOMAC Pain and WOMAC Physical Function at first assessment (week 2) versus placebo (WOMAC Pain mean change from baseline [SE] −2.20 [0.21], −2.87 [0.21], and −2.89 [0.21] in the placebo, tanezumab 2.5 mg, and tanezumab 2.5/5 mg groups, respectively, P≤0.01 for each tanezumab arm versus placebo; WOMAC Physical Function mean change [SE] −2.14 [0.21], −2.89 [0.21], and −3.05 [0.21] in the placebo, tanezumab 2.5 mg, and tanezumab 2.5/5 mg groups, respectively, P≤0.001 for each tanezumab arm versus placebo). At week 16, a greater proportion of patients in each tanezumab regimen (54.5% and 57.1% in the tanezumab 2.5 mg and tanezumab 2.5/5 mg groups, respectively) reported ≥50%, reduction from baseline in WOMAC Pain subscale compared with placebo (37.9%, P≤0.001 for all). A greater proportion of patients in each tanezumab regimen also reported 30%, and 70%, reduction from baseline in WOMAC Pain compared with placebo at week 16 (P≤0.05 for all); there was no significant difference across treatment groups at the ≥90%, response level at week 16 (data not shown). In general, the majority of patients who did not achieve ≥15%, ≥30%, or ≥50%, reduction from baseline at week 8 also did not respond at week 16. However, of patients who did not achieve a ≥50%, reduction from baseline for WOMAC Pain at week 8, a higher proportion (33%) experienced ≥50%, improvement relative to baseline at week 16 after receiving tanezumab 5 mg at week 8 compared with those receiving another 2.5 mg dose (22%) or those treated with placebo (19%).

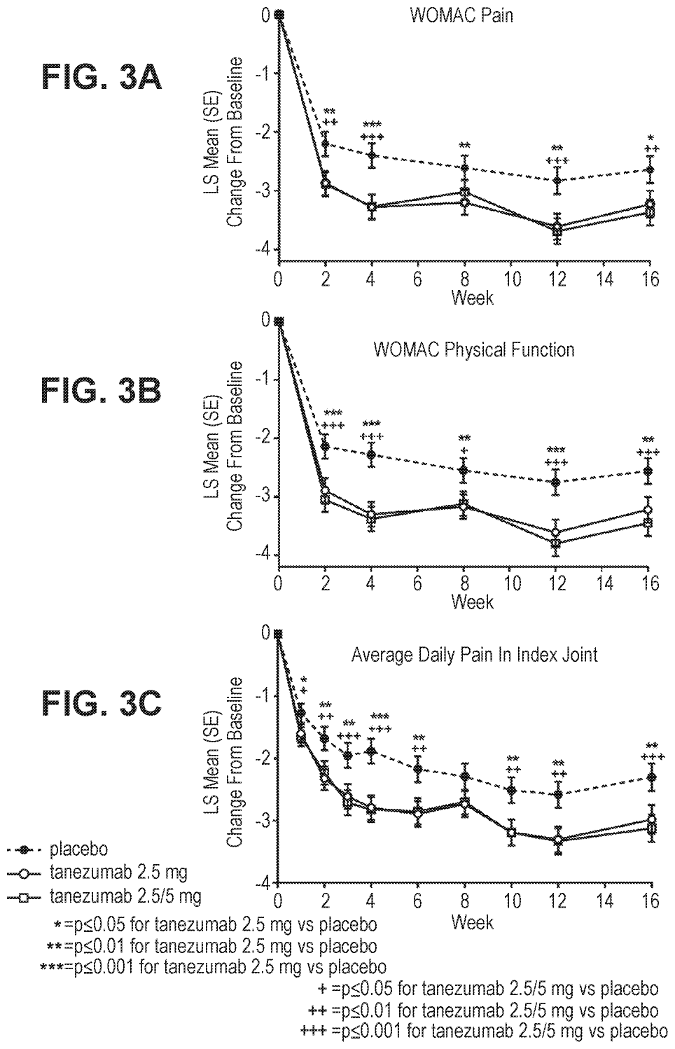

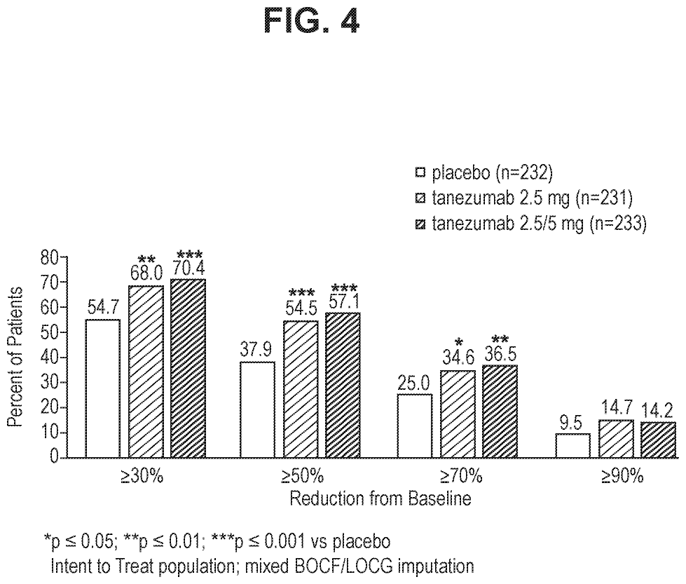

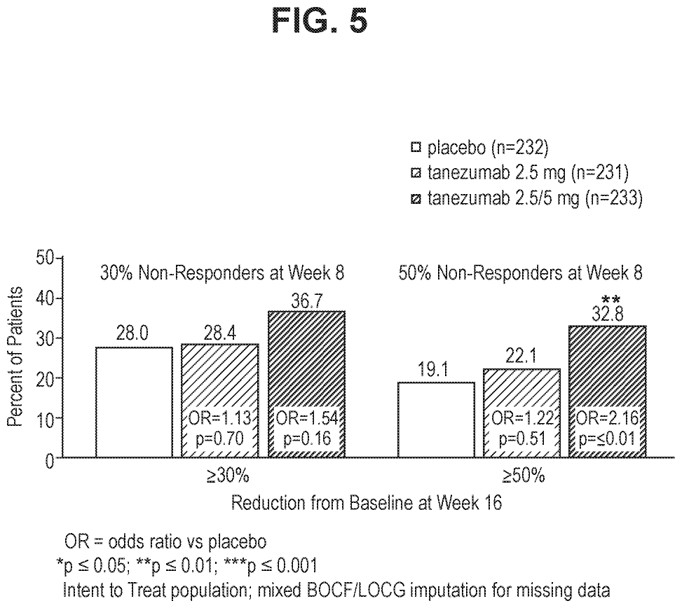

There was a modest benefit of dose titration when the tanezumab 2.5/5 mg group received tanezumab 5 mg after the week 8 efficacy assessments. Of those tanezumab-treated patients with no treatment response (<30% reduction in WOMAC Pain) at the week 8 assessment, up-titration to the 5 mg dose resulted in 22.2% (20/90) transitioning to a moderate response and 18.9% (17/90) to a substantial response at week 12 (the tanezumab 2.5/5 mg group), compared with 11.4% (10/88) and 15.9% (14/88), respectively, in the tanezumab 2.5 mg group who received a second dose of tanezumab 2.5 mg. However, in tanezumab-treated patients already achieving a moderate or substantial response (≥30% reduction in WOMAC Pain) at the week 8 assessment, up-titration to the 5 mg dose did not increase the probability of maintaining a moderate or substantial response at week 12: 59.7% (138/231) of patients in the 2.5 mg group and 59.2% (138/233) of patients in the 2.5/5 mg group maintained a moderate or substantial response from week 8 to week 12. Therefore, non-responders benefited from dose titration while responders did not. At the week 12 and week 16 assessments, efficacy improvements from baseline were modestly greater for the tanezumab 2.5/5 mg group than the tanezumab 2.5 mg group (FIG. 3).

The proportion of patients who took rescue medication was not significantly different between the two tanezumab treatment groups and placebo, except at week 2, in which more placebo-treated patients reported taking rescue medication compared with those treated with tanezumab 2.5/5 mg (P≤05), and at week 4, in which more placebo-treated patients reported taking rescue medication than patients in either tanezumab treatment arm (P≤05 for both; data not shown).

Safety

The frequency of AEs was similar across treatment groups. Most AEs were mild to moderate in severity (data not shown). Nasopharyngitis, pain in extremity, and paresthesia occurred in ≥3% of patients in any treatment group and more frequently in tanezumab-treated patients compared with placebo during the treatment period. Seven patients were discontinued due to AEs. One death due to non-small cell lung cancer stage IV and one due to suicide were reported during the safety follow-up period in the tanezumab 2.5/5 mg group; neither was considered treatment-related.

Neurological Assessments

The incidence of AEs of abnormal peripheral sensation was low across treatment groups, and all were mild or moderate in severity. Most new or worsened abnormalities at the final neurological examination were deemed not clinically significant. There was no substantial difference in Neuropathy Impairment Score change from baseline between tanezumab-treated patients and placebo at any time point (data not shown). No diagnoses of sympathetic neuropathy were reported by the principal investigator in patients evaluated by cardiology or neurology specialists.

Joint Safety Events

Thirty-seven patients had adjudicated joint safety events. Most patients (30/37, 81%) had joint safety events adjudicated as normal OA progression. Adjudicated RPOA cases were classified by the predefined terms: type 1 (accelerated joint space narrowing [≤2 mm]; n=4) or type 2 (damage or deterioration of the joint; n=2), and were seen only in tanezumab-treated patients (6/464; 1.3%). The majority of adjudicated joint safety events were TJRs (28/37 patients; 76%). Most patients underwent TJRs that were of the index joint (26/28 patients; 93%), elective (i.e., there was no associated AE and the TJR was adjudicated as normal OA progression; 21/28 patients; 75%), adjudicated as normal OA progression (26/28 patients; 93%), and occurred after the treatment period (19/28 patients; 68%).

Discussion

Tanezumab demonstrated superior efficacy in both dose arms compared with placebo at week 16 across all 3 co-primary endpoints in this study in which approximately 85% of patients had knee index joints and approximately 15% had hip index joints. Improvements in pain and physical function were significant at the first time-point measured at week 2. Forced titration of tanezumab from 2.5 mg to 5 mg resulted in modest efficacy improvements compared with patients who continued on tanezumab 2.5 mg.

Overall, tanezumab was generally safe and well-tolerated. Across treatment groups, more AEs occurred during the treatment period versus the safety follow-up period. Nasopharyngitis, pain in extremity, and paresthesia were each observed in ≥3% of patients in any treatment group and were more common in tanezumab-treated patients than in placebo-treated patients. The observed pattern for paresthesia and pain in extremity is consistent with data from previous controlled phase 3 tanezumab studies of OA. In the present study, several of the most common AEs overall (eg, arthralgia, paresthesia) were also among the most common AEs in previous tanezumab studies of OA. However, in the present study, arthralgia was less common in tanezumab-treated patients than in placebo-treated patients, a pattern that differed from previous tanezumab studies.

In this study, few patients overall (37/696; 5%) experienced joint safety events that warranted adjudication, and most events (30/37; 81%) were adjudicated as normal OA progression. Overall, RPOA (type 1, accelerated joint space narrowing; type 2, damage or deterioration of the joint) occurred in 6 (1.3%) tanezumab-treated patients (Pivec et al., Orthopedics. 2013; 36(2):118-125). No joint safety events were adjudicated as osteonecrosis, subchondral insufficiency fracture (one case was considered to have been present before the study), or pathologic fracture. Events adjudicated as RPOA (types 1 and 2) occurred more frequently in the tanezumab 2.5 mg group (2.2%) compared with tanezumab 2.5/5 mg (0.4%), suggesting no tanezumab dose-response effect for RPOA in this study. There was no consistent pattern of pain relief or of severe increase in pain associated with RPOA cases. Both RPOA type 2 events occurred in index joints that were Kellgren-Lawrence grade 4 at screening, and neither patient reported NSAID use during the study. In one RPOA type 2 case, the screening x-ray adjudicated after study completion suggested that this patient had atrophic OA and possible osteonecrosis before tanezumab treatment.

All TJRs occurred in joints that were Kellgren-Lawrence grade 3-4 at screening. Most TJRs occurred after the treatment period (68% of patients) and were elective (75% of patients), i.e., the TJR was not associated with an AE and the events were adjudicated as normal OA progression. More TJRs occurred in tanezumab-treated patients than in placebo-treated patients; however, most TJRs were in joints adjudicated as normal OA progression. The higher TJR incidence in tanezumab-treated patients may be explained at least partially by the higher degree of pain relief experienced by these patients; for example, patients who experienced satisfaction with treatment may have been less tolerant of severe pain after washout and less willing to remain in pain. A total of 2 tanezumab-treated patients had TJRs and adjudicated RPOA. The longer observation period in this study may contribute to the higher incidence of TJRs compared with prior tanezumab studies.

The adjudication process together with the long post-treatment observation period allowed for a more comprehensive assessment of OA development in patients treated with tanezumab. However, the short duration and limited study population represent limitations. The 16-week treatment period is too short to assess the ability to maintain efficacy in treating symptomatic OA pain with repeated tanezumab dosing over longer periods. While the study population is appropriate for demonstrating efficacy, larger patient populations studied over longer durations are required for more precise estimates of safety events.

In conclusion, the present study findings suggest that both SC tanezumab 2.5 mg and 2.5/5 mg dosing regimens are generally safe and well-tolerated. Neurological AEs and RPOA incidence were low among tanezumab-treated patients. Although more tanezumab-treated patients underwent TJRs, most TJRs occurred in joints adjudicated as normal OA progression. Moreover, the study efficacy findings suggest that both tanezumab 2.5 mg and 2.5/5 mg may provide significant pain relief and improved function in patients with moderate to severe hip or knee OA who have demonstrated intolerance or incomplete response to standard OA pain treatments.

TABLE 2
Patient Demographic Characteristics and Baseline Measurements

| | | | Tanezumab | | Tanezumab | |
|---|---|---|---|---|---|---|
| | Placebo | | 2.5 mg SC | | 2.5/5 mg SC | |
| | (n = 232) | | (n = 231) | | (n = 233) | |
| Female, no. (%) | 157 | (67.7) | 145 | (62.8) | 151 | (64.8) |
| Age, mean (range), y | 60.4 | (31-85) | 60.9 | (27-84) | 61.2 | (32-83) |
| Race, no. (%) | | | | | | |
| White | 156 | (67.2) | 178 | (77.1) | 170 | (73.0) |
| Black or African American | 60 | (25.9) | 43 | (18.6) | 50 | (21.5) |
| Asian | 13 | (5.6) | 5 | (2.2) | 8 | (3.4) |
| Other | 3 | (1.3) | 5 | (2.2) | 5 | (2.1) |
| History of inadequate pain relief from | | | | | | |
| or intolerance to classes of pain | | | | | | |
| medication, n (%) | | | | | | |
| Acetaminophen/Paracetamol | 232 | (100) | 230 | (99.6) | 232 | (99.6) |
| Inadequate pain relief | 232 | (100) | 230 | (99.6) | 231 | (99.1) |
| Intolerability | 0 | | 0 | | 1 | (0.4) |
| NSAIDs-oral | 232 | (100) | 230 | (99.6) | 233 | (100) |
| Contraindication | 5 | (2.2) | 12 | (5.2) | 7 | (3.0) |
| Inadequate pain relief | 211 | (90.9) | 209 | (90.5) | 211 | (90.6) |
| Intolerability | 23 | (9.9) | 16 | (6.9) | 22 | (9.4) |
| Opioids | 179 | (77.2) | 172 | (74.5) | 180 | (77.3) |
| Contraindication | 1 | (0.4) | 5 | (2.2) | 3 | (1.3) |
| Inadequate pain relief | 58 | (25.0) | 69 | (29.9) | 58 | (24.9) |
| Intolerability | 33 | (14.2) | 28 | (12.1) | 33 | (14.2) |
| Unwilling to take | 90 | (38.8) | 78 | (33.8) | 89 | (38.2) |
| Tramadol | 71 | (30.6) | 73 | (31.6) | 79 | (33.9) |
| Contraindication | 0 | | 1 | (0.4) | 0 | |
| Inadequate pain relief | 62 | (26.7) | 53 | (22.9) | 65 | (27.9) |
| Intolerability | 9 | (3.9) | 19 | (8.2) | 14 | (6.0) |
| Time since OA diagnosis, mean | 9.4 | (0.1-49.9) | 9.5 | (0.0-48.4) | 9.1 | (0.0-52.5) |
| (range), y | | | | | | |
| Index joint, no. (%) | | | | | | |
| Hip | 33 | (14.2) | 34 | (14.7) | 35 | (15.0) |
| Knee | 199 | (85.8) | 197 | (85.3) | 198 | (85.0) |
| WOMAC Pain subscale, mean | 7.3 | (4.2-10.0) | 7.1 | (4.8-10.0) | 7.3 | (5.0-10.0) |
| (range) | | | | | | |
| WOMAC Physical Function subscale, | 7.4 | (4.4-10.0) | 7.2 | (5.1-9.9) | 7.4 | (3.2-9.9) |
| mean (range) | | | | | | |
| Patient's Global Assessment of | | | | | | |
| Osteoarthritis, no. (%) | | | | | | |
| Good (2) | 0 | | 1 | (0.4) | 0 | |
| Fair (3) | 134 | (57.8) | 144 | (62.3) | 125 | (53.6) |
| Poor (4) | 89 | (38.4) | 74 | (32.0) | 92 | (39.5) |
| Very poor (5) | 9 | (3.9) | 12 | (5.2) | 16 | (6.9) |
| Patient's Global Assessment of | 3.46 | (3-5) | 3.42 | (2-5) | 3.53 | (3-5) |
| Osteoarthritis, mean (range) | | | | | | |
| Kellgren-Lawrence grade of index | | | | | | |
| joint, no. (%) | 0 | | 1 | (0.4) | 0 | |
| 1 | 65 | (28.0) | 60 | (26.0) | 59 | (25.4) |
| 2 | 98 | (42.2) | 101 | (43.7) | 105 | (45.3) |
| 3 | 69 | (29.7) | 69 | (29.9) | 68 | (29.3) |
| 4 | | | | | | |

NSAID, nonsteroidal anti-inflammatory drug; OA, osteoarthritis; SC, subcutaneous; WOMAC, Western Ontario and McMasters Universities Osteoarthritis Index.

TABLE 3
Change from Baseline to Week 16 in WOMAC Pain subscale, WOMAC Physical
Function subscale, and Patient's Global Assessment of OA.

| | | | tanezumab | | tanezumab | |
|---|---|---|---|---|---|---|
| | placebo | | 2.5 mg | | 2.5/5 mg | |
| | N = 232 | | N = 231 | | N = 233 | |
| WOMAC Pain^{a} | | | | | | |
| Mean (Range) Baseline Pain Score | 7.30 | (4.2, 10.0) | 7.08 | (4.8, 10.0) | 7.33 | (5.0, 10.0) |
| LS Mean (SE) Change from Baseline | −2.64 | (0.23) | −3.23 | (0.23) | −3.37 | (0.22) |
| Diff of LS Means (SE) | | | −0.60 | (0.24) | −0.73 | (0.24) |
| p-value | | | 0.0129 | | 0.0023 | |
| WOMAC Physical Function^{b} | | | | | | |
| Mean (Range) Baseline Physical | 7.38 | (4.4, 10.0) | 7.18 | (5.1, 9.9) | 7.39 | (3.2, 9.9) |
| Function Score | | | | | | |
| LS Mean (SE) Change from Baseline | −2.56 | (0.22) | −3.22 | (0.22) | −3.45 | (0.22) |
| Diff of LS Means (SE) | | | −0.66 | (0.24) | −0.89 | (0.24) |
| p-value | | | 0.0065 | | 0.0002 | |
| PGA-OA^{c} | | | | | | |
| Mean (Range) Baseline Score | 3.46 | (3, 5) | 3.42 | (2, 5) | 3.53 | (3, 5) |
| LS Mean (SE) Change from Baseline | −0.65 | (0.08) | −0.87 | (0.08) | −0.90 | (0.08) |
| Diff of LS Means (SE) | | | −0.22 | (0.09) | −0.25 | (0.09) |

| | | |
|---|---|---|
| p-value | 0.0109 | 0.0038 |

^{a}WOMAC Pain subscale on an 11-point numerical rating scale; higher score indicates higher pain levels
^{b}WOMAC Physical Function subscale on an 11-point numerical rating scale; higher score indicates worse function
^{c}PGA-OA scale ranges from 1 = “very good” to 5 = “very poor”

### Example 2

### Objectives:

To assess efficacy and safety of tanezumab in patients with moderate to severe OA pain, who have not responded to or cannot tolerate standard of care analgesics.

### Methods:

A randomized, double-blind, placebo-controlled study (24-week treatment; 24-week safety follow-up) was conducted in patients from Europe and Japan with OA of the knee or hip based on American College of Rheumatology criteria. Key inclusion criteria were Western Ontario and McMaster Universities Osteoarthritis Index (WOMAC) Pain and Physical Function scores in the index joint; a Patient Global Assessment of OA (PGA-OA) score of fair, poor, or very poor; a history of insufficient pain relief or intolerance to acetaminophen, oral NSAID, and either tramadol or opioids (or unwilling to take opioids). Patients received subcutaneous tanezumab (2.5 or 5 mg) or placebo at baseline, week 8, and week 16. Co-primary endpoints were change from baseline in WOMAC Pain, WOMAC Physical Function, and PGA-OA scores at week 24 compared with placebo. Three key secondary endpoints were: reduction in the WOMAC Pain subscale of 50%, at Week 24, WOMAC Pain subscale change from Baseline to Week 2, and weekly average pain score (based on daily diary) in the index joint change from Baseline to Week 1. Safety, including adjudication of joint safety events, was assessed.

### Results:

The demographic and baseline characteristics (Table 3) were similar across the study arms.

TABLE 4
Key Demographic and Baseline Characteristics - Safety Population

| | | | tanezumab | | tanezumab | |
|---|---|---|---|---|---|---|
| | placebo | | 2.5 mg | | 5 mg | |
| | (N = 282) | | (N = 283) | | (N = 284) | |
| Age (years) | | | | | | |
| Mean (Range) | 64.2 | (26, 87) | 65.2 | (41, 88) | 65.2 | (32, 89) |
| Sex [n (%)] | | | | | | |
| Male | 86 | (30.5) | 85 | (30.0) | 91 | (32.0) |
| Female | 196 | (69.5) | 198 | (70.0) | 193 | (68.0) |
| Race [n (%)] | | | | | | |
| White | 247 | (87.6) | 245 | (86.6) | 248 | (87.3) |
| Black or African American | 0 | | 0 | | 0 | |
| Asian | 34 | (12.1) | 38 | (13.4) | 34 | (12.0) |
| Other | 1 | (0.4) | 0 | | 2 | (0.7) |
| Years since Index Joint | | | | | | |
| Osteoarthritis Diagnosis | | | | | | |
| Mean (Range) | 7.4 | (0.0, 37.9) | 6.0 | (0.0, 30.6) | 6.7 | (0.0, 37.7) |
| Index Joint [n (%)] | | | | | | |
| Hip | 47 | (16.7) | 49 | (17.3) | 48 | (16.9) |
| Knee | 235 | (83.3) | 234 | (82.7) | 236 | (83.1) |
| Kellgren-Lawrence Grade^{a} of Index | | | | | | |
| Hip or Knee [n (%)] | | | | | | |
| 0 | 0 | | 2 | (0.7) | 0 | |
| 1 | 0 | | 0 | | 0 | |
| 2 | 59 | (20.9) | 49 | (17.3) | 58 | (20.4) |
| 3 | 123 | (43.6) | 131 | (46.3) | 121 | (42.6) |
| 4 | 100 | (35.5) | 101 | (35.7) | 105 | (37.0) |
| WOMAC Pain^{b} at Baseline | | | | | | |
| Mean (Range) | 6.59 | (4.4, 9.4) | 6.70 | (2.8, 10.0) | 6.60 | (4.6, 9.6) |
| WOMAC Physical Function^{c} at | | | | | | |
| Baseline | | | | | | |
| Mean (Range) | 6.67 | (5.1, 9.4) | 6.77 | (4.9, 9.8) | 6.76 | (4.6, 9.4) |
| PGA-OA at Baseline [n (%)] | | | | | | |
| Very Good (1) | 0 | | 0 | | 1 | (0.4) |
| Good (2) | 0 | | 0 | | 1 | (0.4) |
| Fair (3) | 145 | (51.6) | 132 | (46.8) | 136 | (47.9) |
| Poor (4) | 117 | (41.6) | 129 | (45.7) | 129 | (45.4) |
| Very Poor (5) | 19 | (6.8) | 21 | (7.4) | 17 | (6.0) |
| Mean (Range) | 3.55 | (3.0, 5.0) | 3.61 | (3.0, 5.0) | 3.56 | (1.0, 5.0) |

^{a}Kellgren-Lawrence grade is a method of classifying OA severity, ranging from 0 (no OA) to 4 (severe OA)
^{b}WOMAC Pain scores range from 0 (no pain) to 10 (extreme pain); mean of 5 pain questions
^{c}WOMAC Physical Function scores range from 0 (no difficulty) to 10 (extreme difficulty); mean of 17 physical function questions

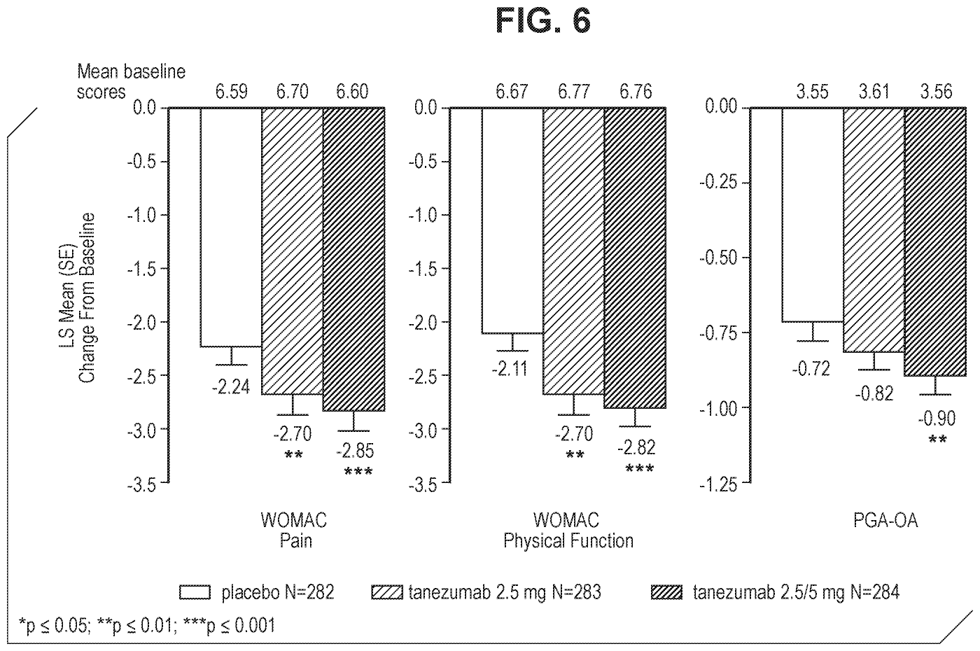

The three co-primary endpoints were change from Baseline to Week 24 in WOMAC Pain, Physical Function, and PGA-OA. Using the step-down testing procedure (equivalent to a single graphical node in gate keeping approach), tanezumab 5 mg treatment provided significantly larger improvements from Baseline than placebo treatment for all 3 co-primary endpoints (Table 5 and FIG. 6). For the tanezumab 2.5 mg treatment, two co-primary endpoints achieved significant improvement, WOMAC Pain (p=0.0088) and WOMAC Physical Function (p=0.0008), but PGA-OA did not achieve significant improvement (p=0.1092) at Week 24. Therefore, no further testing was performed (and drawing of conclusions), particularly with respect to key secondary endpoints.

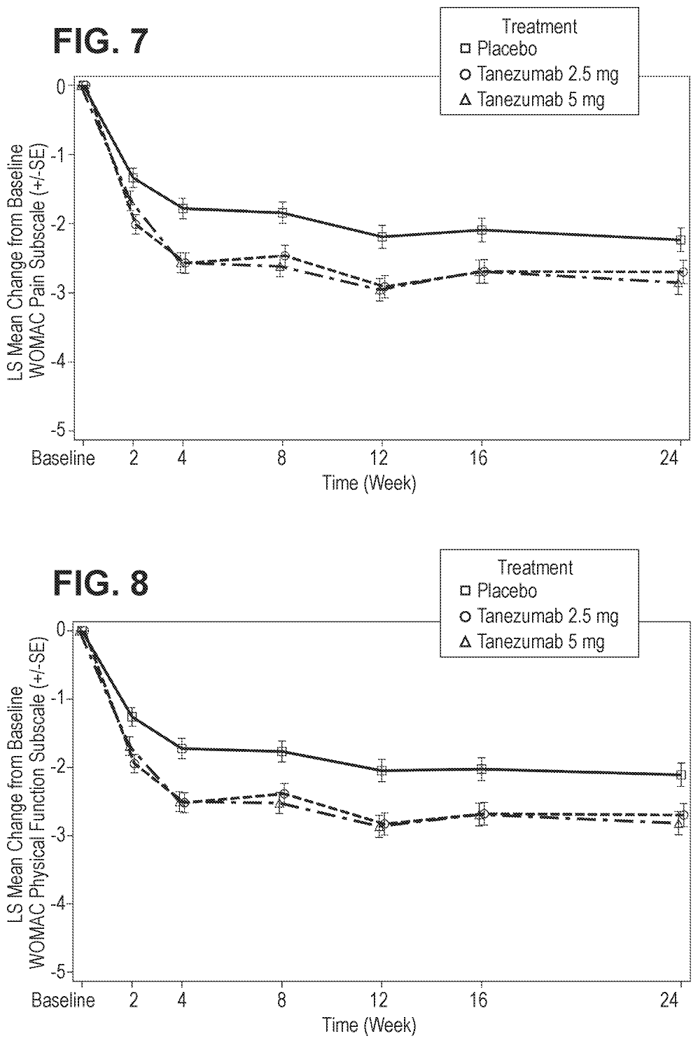

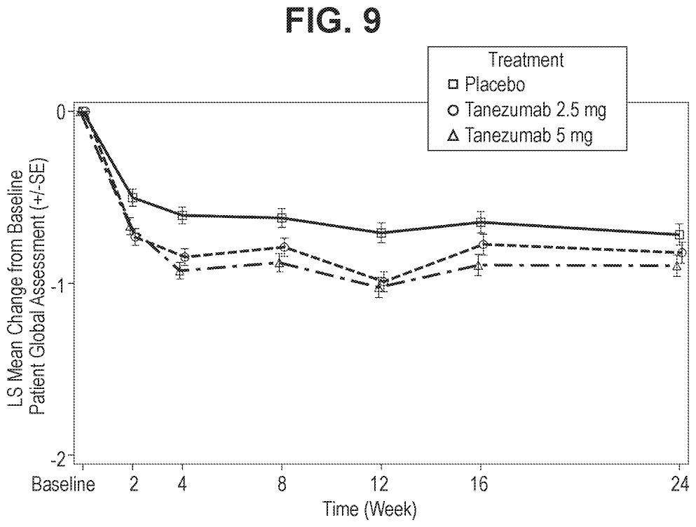

As illustrated in FIG. 7, FIG. 8, and FIG. 9, the change from Baseline for WOMAC Pain and Physical Function were similar over time between the tanezumab 2.5 mg and 5 mg treatment groups, whereas for PGA-OA the tanezumab 5 mg dose treatment group was slightly more improved than for the tanezumab 2.5 mg dose treatment group over time.

TABLE 5
Change from Baseline for Co-Primary Endpoints
at Week 24 (ITT, Multiple Imputation)

| | | tanezumab | tanezumab |
|---|---|---|---|
| | placebo | 2.5 mg | 5 mg |
| | (N = 282) | (N = 283) | (N = 284) |
| WOMAC Pain | | | |
| LS Mean (SE) | −2.24 (0.17) | −2.70 (0.17) | −2.85 (0.17) |
| LS Mean Difference vs. | | −0.46 (0.18) | −0.62 (0.18) |
| placebo (SE) | | | |
| p-value | | 0.0088 | 0.0006 |
| WOMAC Physical | | | |
| Function | | | |
| LS Mean (SE) | −2.11 (0.17) | −2.70 (0.17) | −2.82 (0.17) |
| LS Mean Difference vs. | | −0.59 (0.18) | −0.71 (0.17) |
| placebo (SE) | | | |
| p-value | | 0.0008 | <0001 |
| PGA-OA | | | |
| LS Mean (SE) | −0.72 (0.06) | −0.82 (0.06) | −0.90 (0.06) |
| LS Mean Difference vs. | | −0.11 (0.07) | −0.19 (0.07) |
| placebo (SE) | | | |
| p-value | | 0.1092 | 0.0051 |

ITT = Intent-to-Treat, PGA-OA = Patient's Global Assessment of Osteoarthritis, SE = standard error.
A change from Baseline <0 is an improvement.

The three key secondary endpoints were ≥50%, reduction in the WOMAC Pain subscale at Week 24, WOMAC Pain subscale change from Baseline to Week 2, and weekly average pain score change from Baseline to Week 1. The testing procedure followed the graphical approach provided in the Appendix. Due to the non-significant results of tanezumab 2.5 mg vs. placebo treatment for PGA-OA (p=0.1092), no key secondary endpoints could be tested. Therefore, all key secondary endpoints were concluded to be not significantly better than placebo treatment. However, all key secondary endpoints were numerically better than placebo treatment for both tanezumab treatment groups (Table 6).

TABLE 6
Results of Key Secondary Efficacy Endpoints (ITT)

| | | tanezumab | | tanezumab 5 | |
|---|---|---|---|---|---|
| | placebo | 2.5 mg | | mg | |
| | (N = 282) | (N = 283) | | (N = 284) | |
| Treatment Response: Reduction in the | | | | | |
| WOMAC Pain subscale of ≥50% at Week 24¹ | | | | | |
| Number (%) of subjects with ≥50% | 95 (33.8%) | 128 | (45.4%) | 136 | (47.9%) |
| reduction | | | | | |
| Odds Ratio vs. placebo | | 1.72 | | 1.87 | |
| p-value* | | 0.0022 | | 0.0004 | |
| WOMAC Pain subscale Change from | | | | | |
| Baseline to Week 2² | | | | | |
| LS Mean (SE) | −1.35 (0.14) | −2.02 | (0.14) | −1.69 | (0.14) |
| LS Mean Difference vs. placebo (SE) | | −0.67 | (0.14) | −0.34 | (0.14) |
| p-value* | | <0001 | | 0.0149 | |
| Average pain score in the index joint | | | | | |
| change from Baseline to Week 1² | | | | | |
| LS Mean (SE) | −0.57 (0.11) | −1.06 | (0.11) | −0.93 | (0.11) |
| LS Mean Difference vs. placebo (SE) | | −0.49 | (0.11) | −0.36 | (0.11) |

| | | |
|---|---|---|
| p-value* | <0001 | 0.0009 |

1: Mixed BOCF and LOCF.
2: Multiple Imputation
ITT = Intent-to-Treat,
BOCF = baseline observation carried forward,
LOCF = last observation carried forward
*These are nominal (unadjusted) p-values. Due lack of significance of tanezumab 2.5 mg for PGA-OA (p = 0.1092), the testing procedure was stopped. No key secondary endpoints can be declared as significantly better than placebo treatment.

In addition, at Week 24, the efficacy of tanezumab (both doses) was better than placebo in patients with an index joint of Kellgren-Lawrence (KL) grade 4 for WOMAC Pain (LS mean difference±SE versus placebo for tanezumab 2.5 mg, −0.19±0.23 [nominal P=0.419] for KL2/3 and −0.84±0.28 [nominal P=0.002] for KL4; and for tanezumab 5 mg, −0.32±0.23 [nominal P=0.173] for KL2/3 and −0.98±0.28 [nominal P=0.001] for KL4).

Treatment-emergent AEs occurred in 55%, 53%, and 57% of patients in the placebo, tanezumab 2.5 mg, and tanezumab 5 mg groups, respectively (Table 1). The incidence of serious AEs was higher in both tanezumab groups (2.5 mg=2.8%; 5 mg=3.2%) relative to placebo (1.1%). Discontinuations due to AEs were similar across groups. AEs occurring in ≥3%, of patients in any group, and more frequently in both tanezumab groups relative to placebo, were back pain and OA. TJRs occurred in 6.7%, 7.8%, and 7.0% of patients in the placebo, tanezumab 2.5 mg, and tanezumab 5 mg groups, respectively. Most joint safety events were adjudicated as normal progression of OA (58/79; 73.4%). Pre-specified composite joint safety endpoint events occurred in 0%, 1.8%, and 3.2% of patients in the placebo, tanezumab 2.5 mg, and tanezumab 5 mg groups, respectively. This included 12 patients with rapidly progressive OA (Type 1 n=8; Type 2 n=4), 1 patient with subchondral insufficiency fracture, and 1 patient with primary osteonecrosis.

### Conclusion:

Tanezumab 5 mg significantly improved all 3 co-primary endpoints of pain, physical function, and PGA-OA. Tanezumab 2.5 mg provided significant improvement in pain and physical function but failed to reach significance on PGA-OA. The key secondary efficacy endpoints for both treatment groups were numerically better than the placebo treatment group.

Tanezumab was effective in patients with severe radiographic osteoarthritis, notably in patients with an index joint Kellgren-Lawrence grade 4.

A similar number of total joint replacements (TJRs) were reported in the three treatment groups so there was no difference across treatment groups for the incidence. Joint safety events were more prevalent with tanezumab than placebo.

Tanezumab has potential as a non-opioid option to improve the treatment of signs and symptoms including pain of osteoarthritis, a debilitating, progressive condition.

### Example 3

### Study Design

This study was a randomized, double-blind, active-controlled, multicenter, parallel-group, Phase 3 trial of the safety and efficacy of tanezumab when administered by SC injection for 56 weeks compared to NSAIDs in patients with osteoarthritis (OA) of the knee or hip, based on American College of Rheumatology criteria with x-ray confirmation. Patients had Baseline WOMAC Pain and Physical Function scores and a Baseline PGA-OA of ‘fair,’ ‘poor,’ or ‘very poor.’ Patients were receiving a stable dose of oral NSAID therapy and had documented history indicating that previous treatment for their OA with acetaminophen and either tramadol or opioids (1) had not provided adequate pain relief, or (2) could not be taken by the patient due to contraindication or inability to tolerate (tramadol, opioids), or (3) the patient was unwilling to take (opioids).

Approximately 3000 patients (approximately 1000 per treatment group) were planned for randomization to one of 3 treatment groups in a 1:1:1 ratio, stratified by the factors of index joint (hip, knee), highest Kellgren-Lawrence grade of any knee or hip joint (2, 3, 4), and NSAID treatment during Screening (naproxen, celecoxib, diclofenac).

Patients received a total of seven SC injections, each separated by 8 weeks and daily oral (PO) study medication BID through Week 56. The 3 treatment groups were:
tanezumab 2.5 mg SC and placebo for NSAID BID PO;
tanezumab 5 mg SC and placebo for NSAID BID PO;
placebo for tanezumab SC and NSAID BID PO.

The NSAID was naproxen 500 mg BID, celecoxib 100 mg BID, or diclofenac ER 75 mg BID.

This study was designed with a total (post-randomization) duration of 80 weeks and consisted of three periods: Screening (up to a maximum of 37 days), Double-blind Treatment (56 weeks), and Safety Follow-up (24 weeks) (FIG. 1). The Screening Period included a Washout Period (lasting 2-30 days) if required, and an Initial Pain Assessment Period (7 days prior to Randomization/Baseline; minimum 3 days).

At the Week 16 visit, patients must have had a 30% or greater reduction in WOMAC Pain subscale relative to Baseline in the index joint and a 15% or greater reduction in WOMAC Pain subscale from Baseline at either Week 2, 4 or 8 in order to continue investigational product. Patients who did not meet these response criteria were discontinued from the Treatment Period and entered the Safety Follow-up period.

### Patient Population

The Intent-to-Treat (ITT) Population included all patients who were randomized and received at least one dose of SC study drug. This analysis set was primary for all efficacy endpoints, which were analyzed according to randomization assignment. The Safety Population included all patients who received at least one dose of SC study treatment. This analysis set was primary for all safety endpoints, which were analyzed according to treatment received.

In this study, the ITT and Safety Populations were identical.

Between 20 Aug. 2015 and 8 Aug. 2017, a total of 3021 patients were randomized at 307 centers in the US, Europe, South America, and Asia/Pacific. Altogether, 1008 patients were randomized to tanezumab 2.5 mg, 1005 to tanezumab 5 mg, and 1008 to NSAID. Six, seven, and twelve patients in each of the treatment groups, respectively, were randomized and not treated. Further patient disposition is shown in Table 7 and Table 8.

TABLE 7
Patient Disposition

| | tanezumab | | tanezumab | | | |
|---|---|---|---|---|---|---|
| | 2.5 mg | | 5 mg | | NSAID | |
| Randomized | 1008 | | 1005 | | 1008 | |
| Not treated | 6 | | 7 | | 12 | |
| Safety Population, n (%) | 1002 | (99.4) | 998 | (99.3) | 996 | (98.8) |
| ITT Population, n (%) | 1002 | (99.4) | 998 | (99.3) | 996 | (98.8) |
| Completed Treatment Phase^{a}, n (%) | 447 | (44.6) | 419 | (42.0) | 446 | (44.8) |
| Discontinued Treatment Phase^{a}, n (%) | 555 | (55.4) | 579 | (58.0) | 550 | (55.2) |
| Adverse Event | 74 | (7.4) | 104 | (10.4) | 58 | (5.8) |
| Death | 2 | (0.2) | 3 | (0.3) | 0 | |
| Lost to Follow-Up | 14 | (1.4) | 11 | (1.1) | 11 | (1.1) |
| Withdrawal By Subject | 63 | (6.3) | 62 | (6.2) | 55 | (5.5) |
| Insufficient Clinical Response | 60 | (6.0) | 63 | (6.3) | 91 | (9.1) |
| Protocol Violation | 18 | (1.8) | 31 | (3.1) | 27 | (2.7) |
| Other | 100 | (10.0) | 98 | (9.8) | 88 | (8.8) |
| Patient Meets Protocol Specified Pain | 224 | (22.4) | 207 | (20.7) | 220 | (22.1) |
| Criteria for Discontinuation | | | | | | |
| Completed Study^{ab}, n (%) | 741 | (74.0) | 729 | (73.0) | 757 | (76.0) |
| Discontinued Study^{a}, n (%) | 261 | (26.0) | 269 | (27.0) | 239 | (24.0) |
| Adverse Event | 23 | (2.3) | 22 | (2.2) | 8 | (0.8) |
| Death | 4 | (0.4) | 4 | (0.4) | 0 | |
| Lost to Follow-Up | 25 | (2.5) | 21 | (2.1) | 31 | (3.1) |
| Withdrawal By Subject | 97 | (9.7) | 104 | (10.4) | 100 | (10.0) |
| Insufficient Clinical Response | 19 | (1.9) | 21 | (2.1) | 22 | (2.2) |
| Protocol Violation | 4 | (0.4) | 6 | (0.6) | 4 | (0.4) |
| Other | 89 | (8.9) | 91 | (9.1) | 74 | (7.4) |

^{a}Denominator is number of subjects in the Safety Population;
^{b}Patients completed the study if they completed the safety follow-up period, regardless of whether they completed the treatment phase.

TABLE 8
Patient Disposition for Safety Follow-Up

| | tanezumab | tanezumab | |
|---|---|---|---|
| | 2.5 mg | 5 mg | NSAID |
| Safety Population | 1002 | 998 | 996 |
| Completed Treatment Phase | 447 (44.6) | 419 (42.0) | 446 (44.8) |
| Completed Safety Follow-Up | 422 (42.1) | 386 (38.7) | 414 (41.6) |
| Discontinued Safety Follow-Up | 24 (2.4) | 28 (2.8) | 28 (2.8) |
| Did not enter Safety Follow-Up | 1 (0.1) | 5 (0.5) | 4 (0.4) |
| Discontinued Treatment Phase | 555 (55.4) | 579 (58.0) | 550 (55.2) |
| Completed Safety Follow-Up | 319 (31.8) | 343 (34.4) | 343 (34.4) |
| Discontinued Safety Follow-Up | 115 (11.5) | 128 (12.8) | 102 (10.2) |
| Did not enter Safety Follow-Up | 121 (12.1) | 108 (10.8) | 105 (10.5) |

The demographic and baseline characteristics (Table 9) were similar across the three treatment groups.

TABLE 9
Key Demographic and Baseline Characteristics - Safety Population

| | tanezumab | | tanezumab | | | |
|---|---|---|---|---|---|---|
| | 2.5 mg | | 5 mg | | NSAID | |
| | (N = 1002) | | (N = 998) | | (N = 996) | |
| Age (years) | | | | | | |
| Mean (Range) | 60.3 | (28, 90) | 61.2 | (31, 87) | 60.3 | (28, 88) |
| Sex [n (%)] | | | | | | |
| Male | 365 | (36.4) | 344 | (34.5) | 334 | (33.5) |
| Female | 637 | (63.6) | 654 | (65.5) | 662 | (66.5) |
| Race [n (%)] | | | | | | |
| White | 705 | (70.4) | 712 | (71.3) | 680 | (68.3) |
| Black or African American | 166 | (16.6) | 162 | (16.2) | 186 | (18.7) |
| Asian | 110 | (11.0) | 95 | (9.5) | 99 | (9.9) |
| Other | 21 | (2.1) | 29 | (2.9) | 31 | (3.1) |
| NSAID Cohort [n (%)] | | | | | | |
| Celecoxib | 324 | (32.3) | 325 | (32.6) | 321 | (32.2) |
| Diclofenac | 193 | (19.3) | 191 | (19.1) | 193 | (19.4) |
| Naproxen | 485 | (48.4) | 482 | (48.3) | 482 | (48.4) |
| Years since Index Joint | | | | | | |
| Osteoarthritis Diagnosis | | | | | | |
| Mean (Range) | 8.0 | (0.0, 52.3) | 7.9 | (0.0, 50.4) | 8.1 | (0.0, 44.4) |
| Index Joint [n (%)] | | | | | | |
| Hip | 151 | (15.1) | 148 | (14.8) | 144 | (14.5) |
| Knee | 851 | (84.9) | 850 | (85.2) | 852 | (85.5) |
| Kellgren-Lawrence Grade^{a} | | | | | | |
| of Index Hip or Knee [n (%)] | | | | | | |
| 0 | 0 | | 4 | (0.4) | 1 | (0.1) |
| 1 | 2 | (0.2) | 2 | (0.2) | 3 | (0.3) |
| 2 | 298 | (29.7) | 303 | (30.4) | 291 | (29.2) |
| 3 | 475 | (47.4) | 474 | (47.5) | 476 | (47.8) |
| 4 | 227 | (22.7) | 215 | (21.5) | 225 | (22.6) |
| WOMAC Pain^{b} at Baseline | | | | | | |
| Mean (Range) | 7.01 | (3.6, 10.0) | 7.02 | (1.6, 10.0) | 6.96 | (2.6, 10.0) |
| WOMAC Physical Function^{c} | | | | | | |
| at Baseline | | | | | | |
| Mean (Range) | 7.09 | (1.5, 10.0) | 7.08 | (1.1, 10.0) | 6.99 | (2.4, 10.0) |
| PGA-OA at Baseline [n (%)] | | | | | | |
| Very Good (1) | 1 | (0.1) | 0 | | 1 | (0.1) |
| Good (2) | 5 | (0.5) | 7 | (0.7) | 3 | (0.3) |
| Fair (3) | 557 | (55.7) | 569 | (57.2) | 592 | (59.6) |
| Poor (4) | 381 | (38.1) | 369 | (37.1) | 355 | (35.7) |
| Very Poor (5) | 56 | (5.6) | 50 | (5.0) | 43 | (4.3) |
| Mean (Range) | 3.49 | (1, 5) | 3.46 | (2, 5) | 3.44 | (1, 5) |

^{a}Kellgren-Lawrence grade is a method of classifying OA severity, ranging from 0 (no OA) to 4 (severe OA);
^{b}WOMAC Pain scores range from 0 (no pain) to 10 (extreme pain); mean of 5 pain questions
^{c}WOMAC Physical Function scores range from 0 (no difficulty) to 10 (extreme difficulty); mean of 17 physical function questions

### Efficacy: Key Results & Supportive Findings

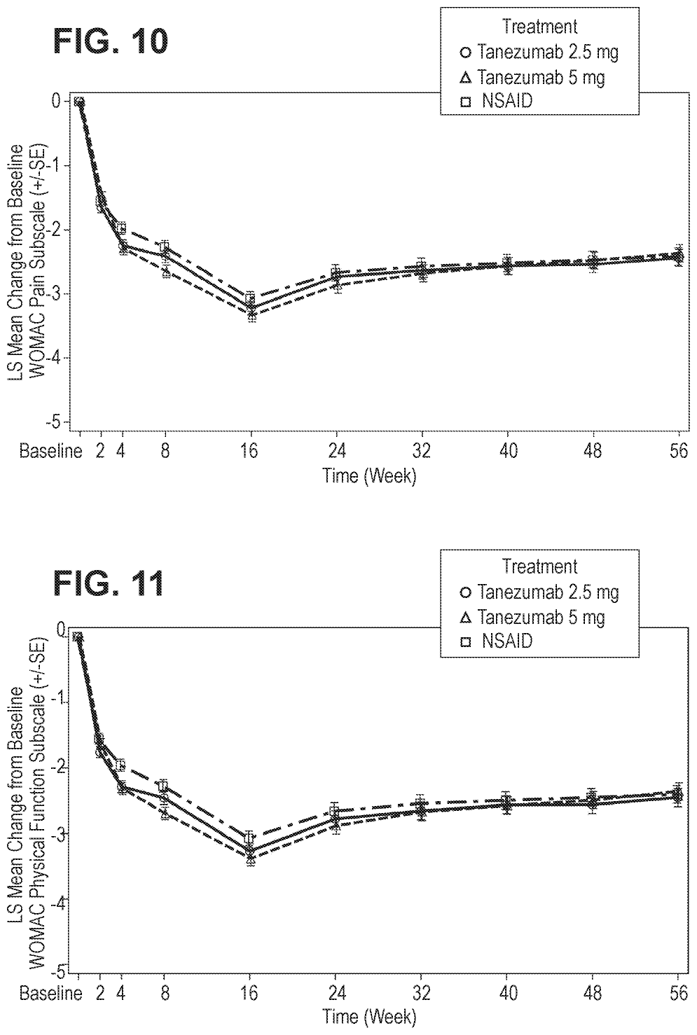

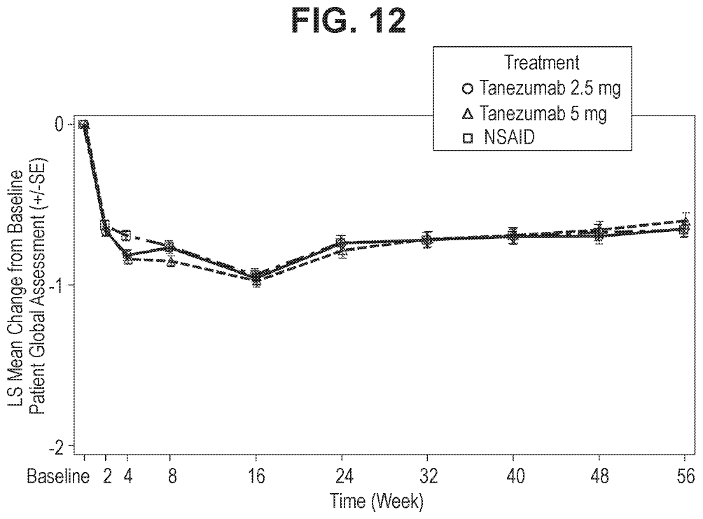

The three co-primary endpoints were change from Baseline to Week 16 in WOMAC Pain, WOMAC Physical Function, and PGA-OA. For the tanezumab 5 mg treatment, two co-primary endpoints achieved significant improvement, WOMAC Pain (p=0.0148) and WOMAC Physical Function (p=0.0030), but PGA-OA did not achieve significant improvement (p=0.3431) at Week 16 (Table 10). Therefore, under the specified testing procedure, no further hypothesis testing (and drawing of conclusions) was performed, particularly with respect to key secondary endpoints. See also FIG. 10, FIG. 11, and FIG. 12.

TABLE 10
Change from Baseline for Co-Primary Endpoints
at Week 16 (ITT, Multiple Imputation)

| | tanezumab | tanezumab | |
|---|---|---|---|
| | 2.5 mg | 5 mg | NSAID |
| | (N = 1002) | (N = 998) | (N = 996) |
| WOMAC Pain | | | |
| LS Mean (SE) | −3.22 (0.11) | −3.33 (0.11) | −3.07 (0.11) |
| LS Mean Difference vs. | −0.15 (0.11) | −0.26 (0.11) | |
| NSAID (SE) | | | |
| p-value | 0.1597 | 0.0148 | |
| WOMAC Physical | | | |
| Function | | | |
| LS Mean (SE) | −3.27 (0.11) | −3.39 (0.11) | −3.08 (0.11) |
| LS Mean Difference vs. | −0.19 (0.11) | −0.31 (0.10) | |
| NSAID (SE) | | | |
| p-value | 0.0691 | 0.0030 | |
| PGA-OA | | | |
| LS Mean (SE) | −0.96 (0.04) | −0.97 (0.04) | −0.94 (0.04) |
| LS Mean Difference vs. | −0.02 (0.04) | −0.04 (0.04) | |
| NSAID (SE) | | | |
| p-value | 0.6332 | 0.3431 | |

ITT = Intent-to-Treat,
OA = osteoarthritis,
SE = standard error
A change from baseline <0 is an improvement.

The key secondary endpoint was 50%, improvement in the WOMAC Pain subscale at Week 16. Due to the non-significant results of tanezumab 5 mg vs. NSAID treatment for PGA-OA, the key secondary endpoint could not be tested. Therefore, the key secondary endpoint was concluded to be not significantly better than NSAID treatment. However, the key secondary endpoint was numerically better than NSAID treatment for both tanezumab treatment groups (Table 11).

TABLE 11
WOMAC Pain Subscale Response: ≥50% Reduction
from Baseline at Week 16 (ITT, Mixed BOCF/LOCF)

| | tanezumab | tanezumab | |
|---|---|---|---|
| | 2.5 mg | 5 mg | NSAID |
| | (N = 1002) | (N = 998) | (N = 996) |
| Number (%) of patients with ≥50% reduction | 549 (54.9%) | 562 (56.5%) | 512 (51.5%) |
| Odds Ratio vs. NSAID (95% CI) | 1.15 (0.96, 1.37) | 1.22 (1.02, 1.46) | |
| p-value¹ | 0.1322 | 0.0262 | |

ITT = Intent-to-Treat,
BOCF = baseline observation carried forward,
LOCF = last observation carried forward,
CI = confidence interval
¹These are nominal (unadjusted) p-values. The testing strategy followed the graphical approach. Due to lack of significance of tanezumab 5 mg for PGA-OA, the testing procedure was stopped. No key secondary endpoints can be declared as significantly better than NSAID treatment.

Other levels of improvement (30%, 70%, and 90%) in the WOMAC Pain subscale at Week 16 are shown in Table 12. The tanezumab 5 mg treatment group exhibited significant improvements at the 70% and 90% level compared to the NSAID treatment group based on unadjusted p-values.

TABLE 12
WOMAC Pain Subscale Response: ≥30%, ≥70%, and ≥90% Reduction
from Baseline at Week 16 (ITT, Mixed BOCF/LOCF)

| | tanezumab | tanezumab | |
|---|---|---|---|
| | 2.5 mg | 5 mg | NSAID |
| | (N = 1002) | (N = 998) | (N = 996) |
| Number (%) of patients with ≥30% reduction | 718 (71.8%) | 725 (72.9%) | 685 (68.9%) |
| Odds Ratio vs. NSAID (95% CI) | 1.15 (0.95, 1.39) | 1.21 (1.00, 1.47) | |
| p-value | 0.1635 | 0.0529 | |
| Number (%) of patients with ≥70% reduction | 289 (28.9%) | 348 (35.0%) | 286 (28.8%) |
| Odds Ratio vs. NSAID (95% CI) | 1.00 (0.83, 1.22) | 1.33 (1.10, 1.61) | |
| p-value | 0.9805 | 0.0033 | |
| Number (%) of patients with ≥90% reduction | 103 (10.3%) | 126 (12.7%) | 84 (8.5%) |
| Odds Ratio vs. NSAID (95% CI) | 1.24 (0.92, 1.69) | 1.57 (1.17, 2.11) | |
| p-value | 0.1590 | 0.0024 | |

ITT = Intent-to-Treat,
BOCF = baseline observation carried forward,
LOCF = last observation carried forward,
CI = confidence interval
p-values are nominal (unadjusted).

Table 13 below summarizes the analysis results of change from Baseline at Week 56 for WOMAC Pain, WOMAC Physical Function, and PGA-OA. Also see FIG. 10, FIG. 11, and FIG. 12 for the time course of the treatment response. There were no notable treatment differences between tanezumab and NSAID treatment groups for change from Baseline at Week 56.

TABLE 13
Change from Baseline for WOMAC Pain, WOMAC Physical Function,
and PGA-OA at Week 56 (ITT, Multiple Imputation)

| | tanezumab 2.5 | | tanezumab 5 | | |
|---|---|---|---|---|---|
| | mg | | mg | | NSAID |
| | (N = 1002) | | (N = 998) | | (N = 996) |
| WOMAC Pain | | | | | |
| LS Mean (SE) | −2.44 | (0.13) | −2.37 | (0.13) | −2.42 (0.14) |
| LS Mean Difference vs. | −0.02 | (0.14) | 0.05 | (0.14) | |
| NSAID (SE) | | | | | |
| p-value | 0.8782 | | 0.7076 | | |
| WOMAC Physical | | | | | |
| Function | | | | | |
| LS Mean (SE) | −2.45 | (0.14) | −2.36 | (0.13) | −2.41 (0.14) |
| LS Mean Difference vs. | −0.05 | (0.14) | 0.05 | (0.14) | |
| NSAID (SE) | | | | | |
| p-value | 0.7305 | | 0.7330 | | |
| PGA-OA | | | | | |
| LS Mean (SE) | −0.65 | (0.05) | −0.60 | (0.05) | −0.66 (0.05) |
| LS Mean Difference vs. | 0.01 | (0.05) | 0.06 | (0.05) | |
| NSAID (SE) | | | | | |

| | | |
|---|---|---|
| p-value | 0.8856 | 0.2814 |

ITT = Intent-to-Treat,
OA = osteoarthritis,
SE = standard error
A change from baseline <0 is an improvement. p-values are nominal (unadjusted).

### Safety

The safety population consisted of 1002 patients who were treated with tanezumab 2.5 mg, 998 treated with tanezumab 5 mg, and 996 treated with NSAID. The largest proportions of patients received 2 doses of SC study medication (31.8%, 30.4%, and 33.5% of patients in the tanezumab 2.5 mg, tanezumab 5 mg, and NSAID treatment groups, respectively) or 7 doses (46.3%, 43.7%, and 44.9%, respectively).

Table 14 summarizes treatment-emergent adverse events during the treatment period. Adverse events were reported by a greater proportion of patients in the tanezumab 5 mg treatment group (67.1%) than in the tanezumab 2.5 mg treatment group (62.8%), while the proportion of patients with adverse events was lowest in the NSAID treatment group (60.3%).

TABLE 14
Incidence of Treatment-Emergent Adverse Events During the
Treatment Period (All Causalities) - Safety Population

| | tanezumab | tanezumab | |
|---|---|---|---|
| | 2.5 mg | 5 mg | NSAID |
| | (N = 1002) | (N = 998) | (N = 996) |
| Number (%) of patients | | | |
| Adverse Event | 629 (62.8) | 670 (67.1) | 601 (60.3) |
| Serious Adverse Event | 51 (5.1) | 80 (8.0) | 46 (4.6) |
| Severe Adverse Event | 45 (4.5) | 68 (6.8) | 45 (4.5) |
| Discontinued from study | 23 (2.3) | 20 (2.0) | 7 (0.7) |
| Discontinued study drug and | 53 (5.3) | 88 (8.8) | 52 (5.2) |
| continued study | | | |

The most frequent adverse events (≥3% in any treatment group) are shown in Table 15. Arthralgia, nasopharyngitis, osteoarthritis, joint swelling, rapidly progressive osteoarthritis, and headache were reported more frequently in each tanezumab treatment group than in the NSAID treatment group (>1% difference between treatment groups). No events were reported more frequently (>1% difference) in the NSAID treatment group than in both tanezumab treatment groups.

TABLE 15
Incidence of Most Frequent (≥3%) Treatment-
Emergent Adverse Events During the Treatment
Period (All Causalities) - Safety Population

| | tanezumab | tanezumab | |
|---|---|---|---|
| | 2.5 mg | 5 mg | NSAID |
| | (N = 1002) | (N = 998) | (N = 996) |
| Number (%) of Patients | n (%) | n (%) | n (%) |
| Arthralgia | 133 (13.3) | 165 (16.5) | 117 (11.7) |
| Nasopharyngitis | 57 (5.7) | 67 (6.7) | 40 (4.0) |
| Back pain | 34 (3.4) | 55 (5.5) | 35 (3.5) |
| Osteoarthritis | 39 (3.9) | 54 (5.4) | 23 (2.3) |
| Joint swelling | 43 (4.3) | 48 (4.8) | 10 (1.0) |
| Peripheral edema | 19 (1.9) | 43 (4.3) | 17 (1.7) |
| Rapidly progressive osteoarthritis | 18 (1.8) | 41 (4.1) | 4 (0.4) |
| Pain in extremity | 31 (3.1) | 37 (3.7) | 28 (2.8) |
| Paraesthesia | 18 (1.8) | 30 (3.0) | 13 (1.3) |
| Fall | 65 (6.5) | 53 (5.3) | 46 (4.6) |
| Headache | 56 (5.6) | 45 (4.5) | 25 (2.5) |
| Musculoskeletal pain | 43 (4.3) | 41 (4.1) | 37 (3.7) |
| Upper respiratory tract infection | 57 (5.7) | 45 (4.5) | 59 (5.9) |

Bolded values represent the highest value across treatment groups.

A total of 10 deaths were reported in this study. Five deaths were reported during the treatment period (2 patients in tanezumab 2.5 mg treatment group and 3 patients in tanezumab 5 mg treatment group), 3 during the safety follow-up period of the study (2 patients in the tanezumab 2.5 mg treatment group and 1 patient in the tanezumab 5 mg treatment group) and 2 occurred after patient discontinuation from the study (1 patient in the tanezumab 5 mg treatment group and 1 patient in the NSAID treatment group).

Four of the 5 deaths that occurred during the treatment period were due to cardiovascular causes (myocardial infarction or cardiac arrest) and the fifth death was caused by a pulmonary embolism. All five patients had relevant medical history of hypertension and/or coronary artery disease. Two of the 3 deaths that occurred during the safety follow-up period were related to respiratory failure in patients with chronic lung disease and extensive histories of tobacco use. The third death that occurred during the safety follow-up period was due to mixed morphine/codeine toxicity. For the patients who died after study discontinuation, the patient in the tanezumab group had a history of hypertension and died from sudden death (no autopsy information was available); the patient in the NSAID treatment group had a history of hypertension and obesity and died from a cardio-respiratory arrest. None of the deaths were considered to be treatment-related by the study investigator.

The most frequent serious adverse events (≥2 patients in any treatment group) are provided in Table 16. The tanezumab 5 mg treatment group had the highest overall incidence of serious adverse events compared to the tanezumab 2.5 mg and NSAID treatment groups. Osteoarthritis, rapidly progressive OA, and arthralgia were reported more frequently in the tanezumab 5 mg treatment group than in the tanezumab 2.5 mg and NSAID treatment groups (≥0.5% treatment difference).

TABLE 16
Incidence of Most Frequent Treatment-Emergent Serious Adverse
Events During the Treatment Period (All Causalities; ≥2 patients
in any treatment group) - Safety Population

| | tanezumab | tanezumab | |
|---|---|---|---|
| | 2.5 mg | 5 mg | NSAID |
| | (N = 1002) | (N = 998) | (N = 996) |
| | n (%) | n (%) | n (%) |
| Any event | 51 (5.1) | 80 (8.0) | 46 (4.6) |
| Osteoarthritis | 9 (0.9) | 17 (1.7) | 4 (0.4) |
| RPOA | 3 (0.3) | 11 (1.1) | 0 |
| Arthralgia | 4 (0.4) | 9 (0.9) | 0 |
| Subchondral | 1 (0.1) | 4 (0.4) | 2 (0.2) |
| insufficiency fracture | | | |
| Meniscus injury | 1 (0.1) | 3 (0.3) | 1 (0.1) |
| Acute kidney injury | 0 | 2 (0.2) | 0 |
| Cellulitis | 0 | 2 (0.2) | 0 |
| Intervetebral disc | 0 | 2 (0.2) | 1 (0.1) |
| protrusion | | | |
| Osteonecrosis | 0 | 2 (0.2) | 0 |
| Pneumonia | 1 (0.1) | 2 (0.2) | 1 (0.1) |
| Cerebrovascular | 2 (0.2) | 0 | 0 |
| accident | | | |
| Acute myocardial | 1 (1.0) | 1 (0.1) | 3 (0.3) |
| infarction | | | |
| Gastrointestinal | 0 | 0 | 2 (0.2) |
| hemorrhage | | | |

A total of 335 patients had joint safety events that met criteria for adjudication (Table 17 and Table 18). The highest number of patients with events requiring adjudication were in the tanezumab 5 mg treatment group (171 [17.1%]), followed by the tanezumab 2.5 mg treatment group (115 [11.5%]), and the NSAID treatment group (49 [4.9%]). The incidence and observation time-adjusted rates of the primary composite joint safety endpoint (rapidly progressive OA, primary osteonecrosis, subchondral insufficiency fracture, pathologic fracture) were highest in the tanezumab 5 mg treatment group (7.1% and 71.5 events/1000 patient-years) compared to the tanezumab 2.5 mg treatment group (3.8% and 37.4 events/1000 patient-years) and the NSAID treatment group (1.5% and 14.8 events/1000 patient-years). The differences in observation time-adjusted rates between each tanezumab treatment group and the NSAID treatment group were statistically significant (tanezumab 2.5 mg vs. NSAID, p=0.0017; tanezumab 5 mg vs. NSAID, p<0.0001; Table 15).

Of the 124 patients with a primary composite joint safety endpoint across the three treatment groups, the vast majority of the events were rapidly progressive OA type 1 (88 events [71%]) followed by rapidly progressive OA type 2 (18 [15%]) and subchondral insufficiency fracture (17 [14%]). There was 1 event of primary osteonecrosis and 0 events of pathologic fracture observed across the treatment groups. The affected joint for the primary composite endpoint was a knee in 96 patients, a hip in 25 patients, and a shoulder in 3 patients. In 28 of the 124 patients, the primary composite endpoint was associated with a total joint replacement in the affected joint (12 rapidly progressive OA type 1, 11 rapidly progressive OA type 2, 1 primary osteonecrosis, and 4 subchondral insufficiency fracture).

The treatment differences in the primary composite endpoint are primarily driven by increased rates of rapidly progressive OA. For rapidly progressive OA (types 1 and 2 combined), the rates were higher for both tanezumab treatment groups (tanezumab 2.5 mg, 3.2% and 31.4 events/1000 patient-years [p=0.0027]; tanezumab 5 mg, 6.3% and 63.3 events/1000 patient-years [p=<0.0001]) compared to the NSAID treatment group (1.2% and 11.9 events/1000 patient-years). In addition, the rate of rapidly progressive OA type 2 was higher in the tanezumab 5 mg treatment group (1.4% and 13.9 events/1000 patient-years; p=0.0008) compared with the NSAID treatment group (0.1% and 1.0 event/1000 patient-years), whereas the rate difference between the tanezumab 2.5 mg (0.3% and 2.9 events/1000 patient-years) and NSAID treatment groups was not significantly different. The rate differences for subchondral insufficiency fracture between either tanezumab treatment group and the NSAID treatment were not statistically different although they were numerically higher.

TABLE 17
Summary of Patients with Adjudicated Joint Safety
Outcomes, Primary Outcome - Safety Population

| | tanezumab | | tanezumab | | | |
|---|---|---|---|---|---|---|
| | 2.5 mg | | 5 mg | | NSAID | |
| | (N = 1002) | | (N = 998) | | (N = 996) | |
| Patients analyzed by the Adjudication | 115 | (11.5%) | 171 | (17.1%) | 49 | (4.9%) |
| Committee, n (%) | | | | | | |
| Primary Composite Joint Safety Endpoint | 38 | (3.8%) | 71 | (7.1%) | 15 | (1.5%) |
| (1), n (%) [95% CI] | [2.7%, 5.2%] | | [5.6%, 8.9%] | | [0.8%, 2.5%] | |
| Secondary Composite Joint Safety Endpoint | 9 | (0.9%) | 22 | (2.2%) | 5 | (0.5%) |
| (2), n (%) [95% CI] | [0.4%, 1.7%] | | [1.4%, 3.3%] | | [0.2%, 1.2%] | |
| Rapidly Progressive OA, n (%) | 32 | (3.2%) | 63 | (6.3%) | 11 | (1.1%) |
| [95% CI] | [2.2%, 4.5%] | | [4.9%, 8.0%] | | [0.6%, 2.0%] | |
| Rapidly Progressive OA type | 29 | (2.9%) | 49 | (4.9%) | 10 | (1.0%) |
| 1, n (%) [95% CI] | [1.9%, 4.1%] | | [3.7%, 6.4%] | | [0.5%, 1.8%] | |
| Rapidly Progressive OA type | 3 | (0.3%) | 14 | (1.4%) | 1 | (0.1%) |
| 2, n (%) [95% CI] | [0.1%, 0.9%] | | [0.8%, 2.3%] | | [0.0%, 0.6%] | |
| Primary Osteonecrosis, n (%) | 0 | | 1 | (0.1%) | 0 | |
| [95% CI] | [0.0%, 0.4%] | | [0.0%, 0.6%] | | [0.0%, 0.4%] | |
| Pathological Fracture, n (%) | 0 | | 0 | | 0 | |
| [95% CI] | [0.0%, 0.4%] | | [0.0%, 0.4%] | | [0.0%, 0.4%] | |
| Subchondral Insufficiency Fracture, n (%) | 6 | (0.6%) | 7 | (0.7%) | 4 | (0.4%) |
| [95% CI] | [0.2%, 1.3%] | | [0.3%, 1.4%] | | [0.1%, 1.0%] | |
| Not enough information to determine Rapid | 2 | (0.2%) | 0 | | 0 | |
| vs. Normal Progression of OA, n (%) | | | | | | |
| Normal Progression of OA, n (%) | 66 | (6.6%) | 79 | (7.9%) | 27 | (2.7%) |
| Other Joint Outcome, n (%) | 9 | (0.9%) | 21 | (2.1%) | 7 | (0.7%) |

OA = osteoarthritis,
CI = confidence interval
(1) The primary composite joint safety endpoint includes any subject with an adjudicated outcome of primary osteonecrosis, rapidly progressive OA type 1 or type 2, subchondral insufficiency fracture, or pathological fracture.
(2) The secondary composite joint safety endpoint includes any subject with an adjudicated outcome of primary osteonecrosis, rapidly progressive OA type 2, subchondral insufficiency fracture, or pathological fracture.
Primary outcome for each subject is shown, according to the following hierarchy: primary osteonecrosis, rapidly progressive OA type 2, subchondral insufficiency fracture, pathological fracture, rapidly progressive OA type 1, not enough information to determine rapid vs. normal progression of OA, other, normal progression of OA.
Includes adjudicated event up to the end of the safety follow-up period or 26 weeks after the end of the treatment period, whichever is later.

TABLE 18
Summary and Analysis of Observation Time-Adjusted Rates
of Adjudicated Joint Safety Outcomes - Safety Population

| | tanezumab | | tanezumab | | | |
|---|---|---|---|---|---|---|
| | 2.5 mg | | 5 mg | | NSAID | |
| | (N = 1002) | | (N = 998) | | (N = 996) | |
| Patients analyzed by the Adjudication | 115 | (11.5%) | 171 | (17.1%) | 49 | (4.9%) |
| Committee, n (%) | | | | | | |
| Primary Composite Joint Safety Endpoint | 1017 | | 993 | | 1011 | |
| (1), observation time (patient-years) | | | | | | |
| n (x/1000 patient-years) | 38 | (37.4) | 71 | (71.5) | 15 | (14.8) |
| [95% CI] | [27.2, 51.3] | | [56.7, 90.2] | | [8.9, 24.6] | |
| rate difference vs. NSAID [95% CI] | 22.5 | [8.5, 36.6] | 56.7 | [38.4, 74.9] | | |
| p-value | 0.0017 | | <0.0001 | | | |
| Secondary Composite Joint Safety Endpoint | 1026 | | 1008 | | 1014 | |
| (2), observation time (patient-years) | | | | | | |
| n (x/1000 patient-years) | 9 | (8.8) | 22 | (21.8) | 5 | (4.9) |
| [95% CI] | [4.6, 16.9] | | [14.4, 33.1] | | [2.1, 11.8] | |
| rate difference vs. NSAID [95% CI] | 3.8 | [−3.3, 11.0] | 16.9 | [6.8, 27.0] | | |
| p-value | 0.2939 | | 0.0010 | | | |
| Rapidly Progressive OA, observation time | 1018 | | 995 | | 1012 | |
| (patient-years) | | | | | | |
| n (x/1000 patient-years) | 32 | (31.4) | 63 | (63.3) | 12 | (11.9) |
| [95% CI] | [22.2, 44.4] | | [49.5, 81.1] | | [6.7, 20.9] | |
| rate difference vs. NSAID [95% CI] | 19.6 | [6.8, 32.4] | 51.5 | [34.5, 68.5] | | |
| p-value | 0.0027 | | <0.0001 | | | |
| Rapidly Progressive OA | 1020 | | 998 | | 1012 | |
| type 1, observation time (patient-years) | | | | | | |
| n (x/1000 patient-years) | 29 | (28.4) | 49 | (49.1) | 11 | (10.9) |
| [95% CI] | [19.8, 40.9] | | [37.1, 65.0] | | [6.0, 19.6] | |
| rate difference vs. NSAID [95% CI] | 17.6 | [5.4, 29.8] | 38.2 | [23.1, 53.4] | | |
| p-value | 0.0047 | | <0.0001 | | | |
| Rapidly Progressive OA | 1027 | | 1010 | | 1016 | |
| type 2, observation time (patient-years) | | | | | | |
| n (x/1000 patient-years) | 3 | (2.9) | 14 | (13.9) | 1 | (1.0) |
| [95% CI] | [0.9, 9.1] | | [8.2, 23.4] | | [0.1, 7.0] | |
| rate difference vs. NSAID [95% CI] | 1.9 | [−1.9, 5.8] | 12.9 | [5.4, 20.4] | | |
| p-value | 0.3214 | | 0.0008 | | | |
| Primary Osteonecrosis, observation time | 1028 | | 1013 | | 1016 | |
| (patient-years) | | | | | | |
| n (x/1000 patient-years) | 0 | (0) | 1 | (1.0) | 0 | (0) |
| [95% CI] | [NE, NE] | | [0.1, 7.0] | | [NE, NE] | |
| Pathological Fracture, observation time | 1028 | | 1013 | | 1016 | |
| (patient-years) | | | | | | |
| n (x/1000 patient-years) | 0 | (0) | 0 | (0) | 0 | (0) |
| [95% CI] | [NE, NE] | | [NE, NE] | | [NE, NE] | |
| Subchondral Insufficiency Fracture, | 1027 | | 1012 | | 1014 | |
| observation time (patient-years) | | | | | | |
| n (x/1000 patient-years) | 6 | (5.8) | 7 | (6.9) | 4 | (3.9) |
| [95% CI] | [2.6, 13.0] | | [3.3, 14.5] | | [1.5, 10.5] | |
| rate difference vs. NSAID [95% CI] | 1.9 | [−4.2, 8.0] | 3.0 | [−3.4, 9.4] | | |

| | | |
|---|---|---|
| p-value | 0.5394 | 0.3636 |

NE = not estimable,
OA = osteoarthritis,
CI = confidence interval
(1) The primary composite joint safety endpoint includes any subject with an adjudicated outcome of primary osteonecrosis, rapidly progressive OA type 1 or type 2, subchondral insufficiency fracture, or pathological fracture.
(2) The secondary composite joint safety endpoint includes any subject with an adjudicated outcome of primary osteonecrosis, rapidly progressive OA type 2, subchondral insufficiency fracture, or pathological fracture.
Includes adjudicated event up to the end of the safety follow-up period or 26 weeks after the end of the treatment period, whichever is later.

In total 89 patients had a joint safety event adjudicated to rapidly progressive OA type 1 (49 in the tanezumab 5 mg treatment group, 29 in the tanezumab 2.5 mg treatment group, and 11 in the NSAID treatment group). In these 89 patients, there were a total of 94 joints affected by rapidly progressive OA type 1 with 84% (79) of events occurring in a knee (Table 19). A hip was the affected joint for 15% (14) of events and for one event (1%) the affected joint was the shoulder. Ninety (96%) of the 94 affected joints adjudicated to rapidly progressive OA type 1 had radiographic evidence of OA on the Screening x-ray (Kellgren Lawrence [KL] grade 1, n=18; KL grade 2, n=39; KL grade 3, n=33; KL grade 4, n=0). A total joint replacement was associated with rapidly progressive OA type 1 in 14% (13) of the 94 events.

Rapidly progressive OA type 2 occurred in 18 patients (14 in the tanezumab 5 mg treatment group, 3 patients in the tanezumab 2.5 mg treatment group, and 1 patient in the NSAID treatment group) with 20 joints affected in total. Across treatment groups, there were 10 affected knees, 8 affected hips, and 2 affected shoulders. Of the 20 affected joints adjudicated to rapidly progressive OA type 2, 16 (80%) had radiographic evidence of OA at Screening (KL grade 1, n=1; KL grade 2, n=1; KL grade 3, n=6; KL grade 4, n=8). The event of rapidly progressive OA type 2 was associated with a total joint replacement for 11 out of 20 (55%) affected joints.

Seventeen patients had joint safety events adjudicated to subchondral insufficiency fracture (7 in the tanezumab 5 mg treatment group, 6 patients in the tanezumab 2.5 mg treatment group, and 4 patients in the NSAID treatment group). Most joints affected by subchondral insufficiency fracture were knees (14 events [82%]); 15 (88%) affected joints had evidence of radiographic OA on the Screening radiograph.

In total, 172 patients only had joint safety events adjudicated to normal progression of OA (79 in the tanezumab 5 mg treatment group, 66 in the tanezumab 2.5 mg treatment group, and 27 in the NSAID treatment group). There were a total of 213 joints adjudicated to normal progression of OA (152 knees, 57 hips, 2 shoulders and 2 other joints). Altogether 206 of the 213 affected joints adjudicated to normal progression of OA had radiographic evidence of OA on the Screening radiograph (Kellgren Lawrence [KL] grade 1, n=5; KL grade 2, n=29; KL grade 3, n=113; KL grade 4, n=59).

TABLE 19
Summary of Details of Adjudicated Joint Safety
Outcomes - Joint-Level - Safety Population

| | tanezumab | | tanezumab | | | |
|---|---|---|---|---|---|---|
| | 2.5 mg | | 5 mg | | NSAID | |
| | (N = 1002) | | (N = 998) | | (N = 996) | |
| Rapidly Progressive OA type 1, n | 30 | | 53 | | 11 | |
| Associated with TJR, n (%) | | | | | | |
| Yes | 3 | (10.0%) | 8 | (15.1%) | 2 | (18.2%) |
| No | 27 | (90.0%) | 45 | (84.9%) | 9 | (81.8%) |
| Joint affected, n (%) | | | | | | |
| Knee | 26 | (86.7%) | 44 | (83.0%) | 9 | (81.8%) |
| Hip | 4 | (13.3%) | 8 | (15.1%) | 2 | (18.2%) |
| Shoulder | 0 | | 1 | (1.9%) | 0 | |
| Baseline Kellgren-Lawrence Grade of | | | | | | |
| Affected Joint, n (%) | | | | | | |
| Not Available | 0 | | 1 | (1.9%) | 0 | |
| 0 | 1 | (3.3%) | 2 | (3.8%) | 0 | |
| 1 | 6 | (20.0%) | 11 | (20.8%) | 1 | (9.1%) |
| 2 | 11 | (36.7%) | 23 | (43.4%) | 5 | (45.5%) |
| 3 | 12 | (40.0%) | 16 | (30.2%) | 5 | (45.5%) |
| 4 | 0 | | 0 | | 0 | |
| Rapidly Progressive OA type 2, n | 3 | | 16 | | 1 | |
| Associated with TJR, n (%) | | | | | | |
| Yes | 1 | (33.3%) | 9 | (56.3%) | 1 | (100.0%) |
| No | 2 | (66.7%) | 7 | (43.8%) | 0 | |
| Joint affected, n (%) | | | | | | |
| Knee | 2 | (66.7%) | 8 | (50.0%) | 0 | |
| Hip | 1 | (33.3%) | 7 | (43.8%) | 0 | |
| Shoulder | 0 | | 1 | (6.3%) | 1 | (100.0%) |
| Baseline Kellgren-Lawrence Grade of | | | | | | |
| Affected Joint, n (%) | | | | | | |
| Not Available | 0 | | 1 | (6.3%) | 1 | (100.0%) |
| 0 | 0 | | 2 | (12.5%) | 0 | |
| 1 | 0 | | 1 | (6.3%) | 0 | |
| 2 | 0 | | 1 | (6.3%) | 0 | |
| 3 | 1 | (33.3%) | 5 | (31.3%) | 0 | |
| 4 | 2 | (66.7%) | 6 | (37.5%) | 0 | |
| Subchondral Insufficiency Fracture, n | 6 | | 7 | | 4 | |
| Associated with TJR, n (%) | | | | | | |
| Yes | 0 | | 3 | (42.9%) | 1 | (25.0%) |
| No | 6 | (100.0%) | 4 | (57.1%) | 3 | (75.0%) |
| Joint affected, n (%) | | | | | | |
| Knee | 4 | (66.7%) | 6 | (85.7%) | 4 | (100.0%) |
| Hip | 2 | (33.3%) | 1 | (14.3%) | 0 | |
| Shoulder | 0 | | 0 | | 0 | |
| Baseline Kellgren-Lawrence Grade of | | | | | | |
| Affected Joint, n (%) | | | | | | |
| Not Available | 0 | | 0 | | 0 | |
| 0 | 0 | | 1 | (14.3%) | 1 | (25.0%) |
| 1 | 0 | | 1 | (14.3%) | 0 | |
| 2 | 2 | (33.3%) | 4 | (57.1%) | 1 | (25.0%) |
| 3 | 4 | (66.7%) | 1 | (14.3%) | 1 | (25.0%) |

| | | | | |
|---|---|---|---|---|
| 4 | 0 | 0 | 1 | (25.0%) |

OA = osteoarthritis
Includes adjudicated event up to the end of the safety follow-up period or 26 weeks after the end of the treatment period, whichever is later.

Among the 335 total patients who had a joint safety event meeting the criteria for adjudication, a total of 157 patients had a total joint replacement (TJR) during the study observation period (Table 20). There were 53 (5.3%) in the tanezumab 2.5 mg treatment group, 79 (7.9%) in the tanezumab 5 mg treatment group, and 25 (2.5%) in the NSAID treatment group. The rate differences vs. NSAID for TJR were statistically greater for both tanezumab treatment groups. Eighty-five of the patients (54%) with TJRs had at least one TJR associated with an adverse event and/or adjudicated to a composite joint safety event (i.e., the surgery was not considered elective). As described above, of the patients who had a total joint replacement, 12 patients had an adjudication outcome of rapidly progressive OA type 1, 11 patients had an adjudication outcome of rapidly progressive OA type 2, 1 patient had an adjudication outcome of primary osteonecrosis, and 4 patients had an adjudication outcome of subchondral insufficiency fracture. For the remaining 129 patients who had a TJR, their adjudication outcome was not enough information to determine rapid vs. normal progression of OA (n=2), normal progression of OA (n=122), or Other (n=5).

Nineteen (12%) of the 157 patients had two or more TJRs during the observation period for a total of 176 TJRs reported during the observation period. Approximately 82% of the TJRs occurred in affected joints that were KL Grade 3 or 4 at Screening and approximately 70% of the TJRs occurred in an index joint. The joints replaced were the knee (n=102), hip (n=69) and shoulder (n=5).

TABLE 20
Summary of Total Joint Replacements - Safety Population

| | tanezumab | | tanezumab | | | |
|---|---|---|---|---|---|---|
| | 2.5 mg | | 5 mg | | NSAID | |
| | (N = 1002) | | (N = 998) | | (N = 996) | |
| Number (%) of patients with ≥1 | 53 | (5.3%) | 79 | (7.9%) | 25 | (2.5%) |
| reported TJR | | | | | | |
| [95% confidence interval] | [4.0%, 6.9%] | | [6.3%, 9.8%] | | [1.6%, 3.7%] | |
| Observation time-adjusted | | | | | | |
| incidence of patients with ≥1 | | | | | | |
| reported TJR | | | | | | |
| Observation time (patient-years) | 1022 | | 1004 | | 1013 | |
| n (x/1000 patient-years) | 53 | (51.8) | 79 | (78.7) | 25 | (24.7) |
| [95% CI] | [39.6, 67.9] | | [63.1, 98.1] | | [16.7, 36.5] | |
| rate difference vs. NSAID [95% | 27.2 | [10.2, 44.1] | 54.0 | [34.1, 73.8] | | |
| CI] | | | | | | |

| | | |
|---|---|---|
| p-value | 0.0017 | <0.0001 |

TJR = total joint replacement,
CI = confidence interval

### Interpretation of Primary Results

The primary safety objective of the study was to characterize the long-term risk of joint safety events in patients with OA of the knee or hip who received tanezumab 2.5 mg or tanezumab 5 mg SC versus NSAID treatment over the course of 56-weeks of treatment using a composite adjudicated endpoint for joint safety. The observation time-adjusted rate of the primary composite endpoint was 37.4 events/1000 patient-years for the tanezumab 2.5 mg treatment group, 71.5 events/1000 patient-years for the tanezumab 5 mg treatment group, and 14.8 events/1000 patient-years for the NSAID treatment group. The rates for the tanezumab treatment groups were statistically significantly higher than in the NSAID treatment group.

Rates for rapidly progressive OA (types 1 and 2 combined and type 1) were significantly higher for each tanezumab treatment group compared to the NSAID treatment group. The rate of rapidly progressive OA type 2 was significantly higher in the tanezumab 5 mg treatment group compared with the NSAID treatment group.

The primary efficacy objective of the study was not achieved with tanezumab 2.5 or 5 mg. There was statistically significant improvement in the co-primary efficacy endpoints of change from Baseline to Week 16 in WOMAC Pain and WOMAC Physical Function, but not PGA-OA, for the tanezumab 5 mg treatment versus NSAID treatment. There was no statistically significant improvement in any of the co-primary efficacy endpoints for the tanezumab 2.5 mg treatment group versus NSAID treatment at Week 16.

There was some evidence that treatment with tanezumab 5 mg provided superior responder rates (≥50% improvement in the WOMAC Pain at Week 16) compared to NSAID treatment, although this could not be declared statistically significant due to the non-significant result for the co-primary endpoint of PGA-OA.

There were no notable treatment differences for change from Baseline at Week 56 for WOMAC Pain, WOMAC Physical Function, and PGA-OA.

The adverse event data are consistent with previous tanezumab OA studies and no new safety signals were identified.

In embodiments that refer to a method of treatment as described herein, such embodiments are also further embodiments for use in that treatment, or alternatively for the manufacture of a medicament for use in that treatment.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof

It is claimed:

1. A method for treating signs and symptoms of osteoarthritis (OA) in a patient, the method comprising administering to the patient an anti-nerve growth factor (NGF) antibody at a dose of 2.5 mg every 8 weeks via subcutaneous injection; wherein the patient has a history of inadequate pain relief or intolerance to analgesic therapy and the treatment with the anti-NGF antibody effectively improves signs and symptoms of OA by at least 16 weeks after the start of treatment with the anti-NGF antibody.

2. A method for treating signs and symptoms of osteoarthritis (OA) in a patient, the method comprising administering to the patient an anti-nerve growth factor (NGF) antibody at a dose of 5 mg every 8 weeks via subcutaneous injection; wherein the patient has a history of inadequate pain relief or intolerance to analgesic therapy and the treatment with the anti-NGF antibody effectively improves signs and symptoms of OA by at least 16 weeks after the start of treatment with the anti-NGF antibody.

3. The method according to claim 1 or 2, wherein the anti-NGF antibody is tanezumab.

4. The method according to any one of claims 1 to 3, wherein the treatment effectively improves OA signs and symptoms as measured by WOMAC Pain subscale, WOMAC Physical Function subscale and/or Patient Global Assessment of OA (PGA-OA).

5. The method according to any one of claims 1 to 4, wherein the treatment effectively improves signs and symptoms of OA by at least 24 weeks after start of treatment.

6. The method according to any one of claims 1 to 5, wherein the treatment effectively improves signs and symptoms of OA by at least 56 weeks after start of treatment.

7. The method according to any one of the preceding claims, wherein the treatment effectively improves WOMAC Pain, WOMAC Physical Function and/or PGA-OA compared to a baseline value prior to or at start of treatment.

8. The method according to claim 7, wherein the treatment further improves one or more clinical measures selected from a) reduction in WOMAC Pain subscale of 50% at week 16 and/or week 24 of treatment; b) reduction in WOMAC Pain subscale from baseline to week 2 of treatment; or c) reduction in average pain score in index joint from baseline at week 1 of treatment.

9. The method according to any one of the preceding claims, wherein the patient was previously treated with the analgesic therapy prior to administering the anti-NGF antibody.

10. The method according to any one of the preceding claims, wherein the patient is not administered an NSAID during the treatment with the anti-NGF antibody.

11. The method according to any one of the preceding claims, wherein the patient is subjected to radiographic assessment of the osteoarthritic joint prior to starting treatment with the anti-NGF antibody.

12. The method according to any one of the preceding claims, wherein the patient is subjected to radiographic assessment of the osteoarthritic joint during treatment with the anti-NGF antibody.

13. The method according to claim 11 or 12, wherein if radiographic assessment identified rapidly progressive osteoarthritis of the joint, the patient is excluded from the treatment with the anti-NGF antibody.

14. The method according to any one of the preceding claims, wherein the patient has moderate to severe osteoarthritis pain.

15. The method according to any one of the preceding claims, wherein the patient, prior to administering the anti-NGF antibody, has a) WOMAC Pain subscale measure of in the osteoarthritic joint; b) WOMAC Physical Function subscale measure of in the osteoarthritic joint; and/or c) a PGA-OA measure of fair, poor, or very poor.

16. The method according to any one of the preceding claims, wherein the patient, prior to administering the anti-NGF antibody, has a Kellgren-Lawrence x-ray grade of ≥2.

17. The method according to any one of the preceding claims, wherein the method further comprises conducting a radiographic assessment of the osteoarthritic joint at regular intervals.

18. The method according to any one of claim 1, or claims 3 to 17 when dependent on claim 1, wherein the 2.5 mg dose is increased to 5 mg after at least one eight week dose.

19. The method according to any one of the preceding claims, wherein the anti-NGF antibody is administered for at least two or more doses at eight weekly intervals.

20. The method according to any one of the preceding claims, wherein the OA is of the hip, knee, shoulder or hand.

21. The method according to any one of the preceding claims, wherein the treatment with the anti-NGF antibody averts opioid addiction in the patient.

22. The method according to any one of the preceding claims, wherein the analgesic therapy comprises the administration of an opioid to the patient.

23. The method according to any one of the preceding claims, wherein the analgesic therapy comprises the administration of tramadol to the patient.

24. The method according to any one of claims 1 to 21, wherein the analgesic therapy comprises the administration of an NSAID to the patient.

25. The method according to any one of the preceding claims, wherein the anti-NGF antibody comprises three CDRs from the variable heavy chain region having the sequence shown in SEQ ID NO: 1 and three CDRs from the variable light chain region having the sequence shown in SEQ ID NO: 2.

26. The method according to any one of the preceding claims, wherein the anti-NGF antibody comprises a HCDR1 having the sequence shown in SEQ ID NO:3, a HCDR2 having the sequence shown in SEQ ID NO:4, a HCDR3 having the sequence shown in SEQ ID NO:5, a LCDR1 having the sequence shown in SEQ ID NO:6, a LCDR2 having the sequence shown in SEQ ID NO:7, and a LCDR3 having the sequence shown in SEQ ID N0:8.

27. The method according to any one of the preceding claims, wherein the anti-NGF antibody comprises a variable heavy chain region having the sequence shown in SEQ ID NO: 1 and a variable light chain region having the sequence shown in SEQ ID NO: 2.

28. The method according to any one of the preceding claims, wherein the anti-NGF antibody comprises a heavy chain having the sequence shown in SEQ ID NO: 9 and a light chain having the sequence shown in SEQ ID NO: 10, wherein the C-terminal lysine (K) of the heavy chain amino acid sequence of SEQ ID NO: 9 is optional.

29. The method according to any one of the preceding claims, wherein the anti-NGF antibody comprises a heavy chain having the sequence shown in SEQ ID NO: 11 and a light chain having the sequence shown in SEQ ID NO: 10.


SEQUENCE LISTING (11 sequences)

SEQ ID NO: 1 - protein, 120 aa, Artificial Sequence
  Synthetic Construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVS

SEQ ID NO: 2 - protein, 109 aa, Artificial Sequence
  Synthetic Construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRT

SEQ ID NO: 3 - protein, 10 aa, Artificial Sequence
  Synthetic Construct
GFSLIGYDLN

SEQ ID NO: 4 - protein, 16 aa, Artificial Sequence
  Synthetic Constuct
IIWGDGTTDYNSAVKS

SEQ ID NO: 5 - protein, 13 aa, Artificial Sequence
  Synthetic Construct
GGYWYATSYYFDY

SEQ ID NO: 6 - protein, 11 aa, Artificial Sequence
  Synthetic Construct
RASQSISNNLN

SEQ ID NO: 7 - protein, 7 aa, Artificial Sequence
  Synthetic Construct
YTSRFHS

SEQ ID NO: 8 - protein, 9 aa, Artificial Sequence
  Synthetic Construct
QQEHTLPYT

SEQ ID NO: 9 - protein, 447 aa, Artificial Sequence
  Synthetic Construct
  MISC_FEATURE (447)..(447): C-terminal lysine (K) at position 447 is optional
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 214 aa, Artificial Sequence
  Synthetic Construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, Artificial Sequence
  Synthetic Construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG